(19) United States
(12) Patent Application Publication
(10) Pub. No.: US 2019/0100761 A1
(43) Pub. Date: Apr. 4, 2019

(54) COMPOSITIONS AND METHODS FOR ENHANCED GENE EXPRESSION AND VIRAL REPLICATION

(71) Applicant: Duke University, Durham, NC (US)

(72) Inventors: Bryan R. Cullen, Durham, NC (US); Edward M. Kennedy, Durham, NC (US); Hal P. Bogerd, Durham, NC (US); Anand Kornepati, Durham, NC (US); Nicholas Heaton, Durham, NC (US); David Courtney, Durham, NC (US)

(73) Assignee: Duke University, Durham, NC (US)

(21) Appl. No.: 16/091,822

(22) PCT Filed: Apr. 6, 2017
(86) PCT No.: PCT/US17/26391

§ 371 Date: Oct. 5, 2018

Related U.S. Application Data
(60) Provisional application No. 62/318,868, filed on Apr. 6, 2016.
(60) Provisional application No. 62/361,282, filed on Jul. 12, 2016.

(51) Int. Cl. C12N 15/67; C12N 7/00; C07K 14/47

(52) U.S. Cl. CPC C12N 15/67; C12N 2740/16052; C07K 14/47; C12N 7/00

(57) ABSTRACT

The invention generally relates to compositions (including polynucleotides, constructs, fusion proteins, vectors, and cells) and methods of using such compositions for enhancing gene expression, protein production and viral replication. More specifically, the invention relates to use of m⁶A sequences and/or YTHDF polypeptides to enhance gene expression or viral replication.

### CROSS-REFERENCE TO RELATED PATENT APPLICATIONS

The present application claims the benefit of priority to U.S. Provisional Patent Application No. 62/318,868, filed on Apr. 6, 2016, and U.S. Provisional Patent Application No. 62/361,282, filed on Jul. 12, 2016 the contents of which are incorporated herein by reference in their entireties.

### STATEMENT REGARDING FEDERALLY SPONSORED RESEARCH

This invention was made with government support under grant number R01-AI117780 awarded by the National Institute of Health. The United States government has certain rights in the invention.

### FIELD OF INVENTION

The invention generally relates to compositions (including constructs, fusion proteins, vectors, and cells) and methods of using such compositions for enhancing gene expression and viral replication. More specifically, the invention relates to use of m⁶A sequences and/or YTHDF polypeptides to enhance gene expression or viral replication.

### INTRODUCTION

Gene expression is the process by which information from a gene is used in the synthesis of a functional gene product such as a protein. The central dogma of molecular biology dictates that information is generally transferred from DNA to RNA to protein, although exceptions have been well documented.

In the biotechnology industry, it is often desirable to enhance RNA expression. For example, recombinant DNA technology is widely used in the biotechnology industry to produce proteins that may be used as research tools, industrial enzymes, or active ingredients in therapeutics. Generally, in such applications, recombinant DNA technology involves the cloning of a gene encoding a desired polypeptide into a suitable expression vector. The expression vector encoding the desired polypeptide is then transfected into a host cell, which is cultured to produce RNA encoding the polypeptide. The RNA is translated to produce the polypeptide. The polypeptide may then be purified either by lysing the cells or, in the case of a secreted polypeptide, purified from the supernatant of the cell culture. Given this methodology, one potential way of increasing the productivity of a cell line producing a desired polypeptide is by increasing the steady-state levels of mRNA encoding the polypeptide in a cell.

In addition to the recombinant production of commercially-valuable proteins, enhanced RNA expression may also be beneficial in other applications such as gene therapy, RNA-based therapeutics (i.e., mRNA-based therapeutics), and virus production. There, however, remains a need in the art for new strategies and mechanisms for enhancing RNA expression in a particular cell or cell line.

### SUMMARY

The invention generally relates to compositions (including polynucleotides, constructs, fusion proteins, vectors, and cells) and methods of using such compositions for enhancing gene expression, protein production and viral replication. More specifically, the invention relates to use of m⁶A sequences and/or YTHDF polypeptides to enhance gene expression or viral replication.

In one aspect, polynucleotides are provided. The polynucleotides may include at least one m⁶A sequence such as, for example, one engineered m⁶A sequence. In another aspect, constructs are provided. The constructs may include a promoter operably connected to any one of the polynucleotides described herein. Alternatively, the constructs may include an insert site, and a UTR sequence including at least one m⁶A sequence. The insert site may or may not include a heterologous coding sequence encoding a heterologous polypeptide.

In another aspect, vectors including any of the polynucleotides or constructs described herein are provided. In another aspect, cells including any of the polynucleotides, constructs, or vectors described herein are provided.

In another aspect, methods for producing a heterologous polypeptide in a cell are provided. The methods may include introducing any of the polynucleotides, constructs, or vectors described herein into the cell.

In a further aspect, provided herein are fusion proteins (and constructs encoding such fusion proteins) including a YTHDF polypeptide and a RNA-binding polypeptide. Constructs including (i) a heterologous coding sequence encoding a heterologous polypeptide, and (ii) a UTR sequence including at least one RNA-binding polypeptide recognition sequence are also provided as are cells including such fusion proteins and constructs.

In another aspect, methods for producing a heterologous polypeptide in a cell including introducing or expressing the fusion proteins (or constructs encoding such fusion proteins) described herein in the cell and introducing or expressing constructs including (i) a heterologous coding sequence encoding a heterologous polypeptide, and (ii) a UTR sequence including at least one RNA-binding polypeptide recognition sequence.

In a still further aspect, cells engineered to overexpress a YTHDF polypeptide as well as methods of using such cells to produce a virus containing at least one m⁶A sequence in a cell are also provided.

### BRIEF DESCRIPTION OF THE DRAWINGS

FIG. 1 shows m⁶A site discovery in HIV-1 isolate NL4-3. FIG. 1A is an overview of the general PAR-CLIP experimental design. FIG. 1B shows schematic of the PA-m⁶A-seq and PAR-CLIP site discovery strategy is depicted. A typical transcript containing an m⁶A editing site is shown with an incorporated adjacent 4SU molecule (orange star). Upon binding, the m⁶A specific antibody or a host YTHDF reader protein is crosslinked to the 4SU. T>C transitions are generated from crosslinked 4SU during reverse transcription/cDNA synthesis. FIG. 1C shows PA-m⁶A-seq and PAR-CLIP were performed 64 h after infection with VSV-G pseudotyped HIV-1 strain NL4-3. Shown are the entire genome coverage tracks for PA-m⁶A-seq in CEM-SS cells, and then the FLAG-GFP control and YTHDF1, 2 and 3 tracks in 293T cells. FIG. 1D shows an expanded view of the 3′UTR region of HIV-1 containing the detected m⁶A editing sites. This ˜1.4 kb region extends from the second coding exon of Rev to the end of the R region. Red/Blue bars indicate sites of T>C conversions. Reads are aligned to an HIV-1 genome that begins with the U5 region and ends with U3-R to avoid repeat alignments. The PA-m⁶A-seq has a Y axis of 0-200 reads, and all others are depicted with Y axes of 0-900 reads.

FIG. 2 shows m⁶A site discovery using primary HIV-1 isolates BaL and JR-CSF. FIG. 2A shows YTHDF1 or YTHDF2 PAR-CLIP binding clusters were mapped for HIV-1 isolates NL4-3, BaL and JR-CSF for the 3′ region of the HIV-1 genome from the second exon of Rev to the end of the R region, as indicated. The three novel YTHDF protein binding clusters discovered for these two viruses are annotated below the relevant track. The Y axes for these alignments are NL4-3: 0-900 reads, BaL: 0-2000 reads, and JR-CSF: 0-1500 reads. FIG. 2B shows alignment of two segments from the NL4-3 and BaL genome, with a putative novel methyl receptor adenosine present in BaL shown in red. FIG. 2C is similar to panel B, except aligning two regions of NL4-3 and JR-CSF, with the two novel methyl acceptor adenosines present in JR-CSF indicated.

FIG. 3 shows consensus m⁶A editing sites mapped to the NL4-3 genome. Shown in FIGS. 3A-3D are the 4 mapped YTHDF PAR-CLIP clusters present in NL4-3 with consensus m⁶A sites indicated. Adjacent T to C conversions, that result from 4SU photo-crosslinking (T=blue, C=red), are indicated. Below are the potential viral m⁶A editing sites shown in red, with a black line indicating the nucleotide position in the YTHDF binding cluster relative to the mutated T residue. This figure identifies all sites with a minimal (5′-RAC-3′) m⁶A consensus but this does not demonstrate that all of these A residues are actually modified.

FIG. 4 shows 3′UTR m⁶A sites boost mRNA abundance and protein expression. Dual luciferase indicators were constructed in which the 3′UTR of RLuc in psiCheck2 was replaced by HIV-1 3′UTR sequences in either a wildtype form or with the m⁶A sites listed in FIG. 3 replaced by G residues. The “HIV 3′ UTR” construct contains the entire ˜1.4 kb 3′UTR region of HIV-1, encompassing all four m⁶A clusters, extending from the second coding exon of Rev through the viral poly(A) addition site. The U3/NF-kB/TAR indicator, which contains the viral 3′ UTR from 5′ of the LTR NF-kB repeats again through the viral poly(A) addition site, retains only the U3/NF-kB and TAR m⁶A sites. In FIG. 4A the indicators were transfected into 293T cells and RLuc and internal control FLuc levels assayed at 48 h post-transfection. In FIG. 4B the transfection was performed in 293T cells, as described in (FIG. 4A). Steady state transcript abundance was measured by qRT-PCR for both the internal control FLuc and the m⁶A cluster-containing RLuc mRNAs. RLuc mRNA abundance is shown normalized first to endogenous GAPDH mRNA and then to the control FLuc mRNA. FIG. 4C is similar to FIG. 4A, except these luciferase assays were performed in transfected CEM-SS T-cells. FIG. 4D is similar to FIG. 4B except that this qRT-PCR analysis of FLuc and RLuc mRNA expression levels was performed in transfected CEM-SS T cells. FIG. 4E shows cellular YTHDF PAR-CLIP clusters with 1, 2, 5, or 6 predicted m⁶A editing sites were compared using the same RLuc indicator assay as described in FIG. 4A and FIG. 4C. These clusters were cloned into the 3′UTR of RLuc in a wildtype or mutant form, lacking m⁶A editing sites and RLuc activity determined. FIG. 4F shows YTHDF fusion proteins were constructed where the carboxy-terminal m⁶A binding domain was replaced with the MS2 coat protein, and these were compared to a negative control GFP-MS2 fusion after co-transfection into 293T cells along with a psiCHECK2 dual luciferase vector with and without MS2 binding sites inserted into the RLuc 3′UTR. (FIGS. 4A-4F). Average of from three to six independent experiments with SD indicated.

FIG. 5 shows overexpression of YTHDF m⁶A reader proteins boosts HIV-1 protein and RNA expression. FIGS. 5A and 5B show qRT-PCR was used to quantify the expression level of the dominant spliced HIV-1 mRNA isoforms encoding Rev, Tat or Nef as well as the unspliced genomic RNA (gRNA). Assays were performed at 24 h (FIG. 5A) or 48 h (FIG. 5B) post-infection (hpi) using 293T cells stably overexpressing GFP (Neg) or one of the three YTHDF proteins (Y1 is YTHDF1 etc). Data were normalized to endogenous GAPDH mRNA. FIGS. 5C and 5D show representative Western blots from HIV-1 infection experiments similar to those described in FIGS. 5A and 5B. Infected 293T cells over-expressing GFP (Neg) or one of the YTHDF proteins were lysed at 24 hpi or 48 hpi then probed with an antibody specific for the HIV-1 p24 capsid protein, Nef, the FLAG tag on the overexpressed YTHDF protein or endogenous β-actin. Shown below the respective bands are actin-normalized quantifications. p55 represents uncleaved HIV-1 Gag polyprotein while p24 is the mature viral capsid FIGS. 5E and 5F show quantifications of band intensities from three independent Western experiments, similar to those shown in (FIGS. 5C and 5D), performed at 24 hpi (FIG. 5E) or 48 hpi (FIG. 5F), with SD indicated.

FIG. 6 shows recruitment of YTHDF2 to viral m⁶A editing sites boosts viral replication in CD4+ T cells. FIG. 6A shows a representative growth curve for HIV-1 NL4-3 in control CEM-SS cells, in a CEM-SS sub-clone lacking a functional YTHDF2 gene (Y2-KO) or in a CEM-SS sub-clone overexpressing YTHDF2 (Y2-OE). HIV-1 replication was monitored by p24 ELISA. FIG. 6B is a graph showing the total level of protein recovered from the cell pellets harvested at the indicated time points from the cultures analyzed in FIG. 6A. FIG. 6C is a bar graph showing the average of 3 independent replicate p24 ELISA growth curve experiments at 96 hpi, with significance of differences indicated. FIG. 6D shows a representative Western blot of samples treated as in FIG. 6A at 72 hpi. This Western analyzes the level of intracellular expression of HIV-1 p24, Nef and YTHDF2, with endogenous β-actin used as a loading control. Equal quantities of protein, as determined by BCA analysis, were loaded in each lane. Mock: mock infected culture.

FIG. 7 is related to FIGS. 1 and 2 and shows PAR-CLIP analysis of YTHDF protein binding to the HIV-1 genome. FIG. 7A shows a Western blot analysis of a FLAG-specific immunoprecipitation of lysates of 293T cells expressing FLAG-GFP, FLAG-YTHDF1 (Y1), FLAG-Y2 or FLAG-Y3. The YTHDF proteins are ˜65 kD in size. FIG. 7B shows the results after crosslinking of 4SU residues to bound proteins, the YTHDF proteins were immunoprecipitated and RNase treated before labeling of protein bound RNA oligonucleotides using γ-³²P-ATP. This gel shows that this results in a readily detectable radiolabeled protein band at the predicted ˜65 kD size for all three YTHDF proteins. FIG. 7C is a bar graph showing the percent of HIV-1-specific reads that contain T-to-C mutations, characteristics of a 4SU crosslink, in the PAR-CLIP libraries obtained from 293T cells expressing GFP, YTHDF1, YTHDF2 or YTHDF3 after infection with the indicated HIV-1 isolates. FIG. 7D is a bar graph showing the mean read length for the PAR-CLIP libraries obtained in 293T cells infected with the indicated HIV-1 isolates. These data derive from FIG. 2. FIG. 7E shows fine mapping of PAR-CLIP reads that map to the NL4-3 TAR element for YTHDF1, 2 and 3. As may be observed, many of these extend into U3, thus demonstrating that the 3′ LTR R element is m⁶A modified.

FIG. 8 is related to FIG. 4 and shows analysis of the YTHDF protein function. FIG. 8A is a PAR-CLIP analysis examining reads obtained from transfected 293T cells that map to the psiCheck2-based indicator construct containing the HIV-1 U3/NF-κB/TAR region used in FIG. 4. As may be observed, we readily detect YTHDF2 binding to the TAR region present in this indicator in the wildtype HIV-1 sequence but we do not observe any YTHDF2 binding to either the HIV-1 or RLuc sequences in the indicator plasmid containing HIV-1 sequences in which the viral m⁶A editing sites have been mutated. FIG. 8B shows data are identical to the results shown in FIG. 4C except that they are here normalized to the parental psiCheck2 vector lacking any 3′UTR insert. FIG. 8C shows data that are identical to the results shown in FIG. 4E except that they are here normalized to the parental psiCheck2 vector lacking any 3′UTR insert. FIG. 8D shows immunofluorescence analysis of the subcellular location of full-length FLAG-tagged YTHDF proteins or the FLAG-tagged YTHDF-MS2 fusion proteins used in FIG. 4F, showing that all are expressed equivalently and localized to the cytoplasm.

FIG. 9 is related to FIG. 6 and shows analysis of CD4 and CXCR4 expression on CEM-SS subclones. FIG. 9A is a comparison of the level of CD4 cell surface expression on the parental CEM-SS cells and the Y2-KO and Y2-OE subclones analyzed in FIG. 6. Average of three independent experiments with SD indicated. FIG. 9B is similar to FIG. 9A, except looking at cell surface CXCR4 expression. While both subclones show ˜2-fold less cell surface CXCR4 than wildtype cells, they are closely similar to each other. FIG. 9C shows a representative trace of a cell surface CD4 FACS analysis looking at the parental CEM-SS cell line and the CEM-SS Y2-KO and Y2-OE subclones. FIG. 9D is similar to FIG. 9C, except looking at cell surface CXCR4.

FIG. 10 is related to FIGS. 2 and 3 and shows conservation of HIV-1 m⁶A editing sites. FIG. 10A shows the sequence conservation of the 10 potential m⁶A editing sites in HIV-1 strain NL4-3 identified in FIG. 3. As may be observed, seven of these 10 sites are highly conserved across HIV-1 isolates in subtypes A, B, C and D. FIG. 10B shows the location of potential m⁶A editing sites in the HIV-1 TAR element which are indicated in red/gray.

FIG. 11 is related to FIG. 6 and shows inhibition of HIV-1 replication by the m⁶A inhibitor DAA. FIG. 11A is a panel showing an m⁶A dot blot for mock treated or DAA treated CEM-SS cells. FIG. 11B is a representative Western blot is shown for HIV-1 NL4-3 infection of mock or DAA treated CEM-SS cells harvested at 72 hpi. The Western shown in FIG. 11B is representative of four independent biological experiments. FIG. 11C is a bar graph showing the level of viable CEM-SS cells observed in cultures grown in the absence or presence of 50 micromolar DAA for 72 h. FIG. 11D is a graph measuring the total level of cellular protein, determined by BCA assay, recovered from the cell pellets derived from the cultures analyzed in panel FIG. 11B. Note that the level of protein is similar in the HIV-1 infected cultures in the presence or absence of DAA.

FIG. 12 shows expression of GFP in an AAV vector. An AAV vector encoding GFP was packaged in wildtype 293T cells or in a clonal 293T cell line overexpressing YTHDF2. Viral particles were isolated after cell lysis by sucrose gradient centrifugation and dilutions (1:2, 1:20, 1:200 and 1:2000) used to infect naive 293T cells and GFP expression then analyzed by FACS. Brown: negative control 293T cells, Blue: AAV derived from wildtype 293T Red: AAV derived from YTHDF2 overexpressing 293T cells. As may be observed, the AAV virions produced in the presence of YTHDF2 gave rise to higher levels of GFP expressing cells.

FIG. 13 shows YTHDF2 strongly enhances IAV gene expression. A549 cells were transduced with a lentiviral vector expressing GFP, YTHDF1 or YTHDF2 and then single-cell cloned. FIG. 13A shows expression of YTHDF1 (Y1). This Western was performed using a Y1-specific antibody. FIG. 13B is similar to panel A except that this Western used a Y2-specific antibody. The endogenous Y2 protein is also detected. FIG. 13C shows the results after A549 cells were infected with IAV strain PR8 at an MOI of 1 and cell cultures lysed at the indicated number of hours post-infection (hpi). α Flag detects the ectopically expressed Y1 or Y2 protein, while the upper two lanes show the IAV NS1 and M2 proteins. Actin was used as a loading control. The numbers shown in the α NS1 lane reflect the level of NS1 protein detected in each culture at 24 h post-infection. The data in FIG. 13 were generated with a high MOI of 1.0, using IAV strain PR8 grown in embryonated chicken eggs and titered on MDCK cells, and were performed in the absence of trypsin in the media so no viral spread will occur.

FIG. 14 shows that overexpression of YTHDF2 increased all aspects of influenza A virus (IAV) replication. FIG. 14A shows clonal A549 cell lines expressing ectopic YTHDF1-Flag (Y1) or YTHDF2-Flag (Y2.1 and Y2.2) were generated using lentiviral vectors. Expression of YTHDF1 and 2 was then confirmed by Western using an anti-FLAG monoclonal. FIG. 14B shows results when YTHDF cell lines were infected with IAV strain PR8 at a multiplicity of infection (MOI) of 0.01 and expression of two viral proteins, NS1 and M2, was then assessed by Western at 24, 48 and 72 hours post infection (hpi). YTHDF1 and YTHDF2 expression was detected by anti-Flag. The parental A549 cell line, and an additional A549 line expressing GFP-Flag, were used as control cell lines. FIG. 14C shows quantitative RT-PCR used to determine the mRNA levels of the spliced IAV mRNA encoding the IAV M2 protein at the same time points post-infection. FIG. 14D shows the titer of infectious IAV produced by these A549-derived cell lines at 72 hpi was determined by plaque assay on the cell line MDCK. A significant increase in viral titer was noted when cells expressing ectopic YTHDF2 were tested (*=p<0.05, **=p<0.01).

### DETAILED DESCRIPTION

The present invention generally relates to the inventors' discovery that m⁶A sequences strongly enhance RNA expression in cis. Without being limited by theory, the inventors have found that m⁶A sequences strongly enhance RNA expression by recruiting cellular YTHDF m⁶A “reader” proteins. As a result, inhibition of YTHDF expression was found to inhibit RNA expression and viral replication, while YTHDF overexpression enhanced RNA expression and viral replication.

Like proteins and DNA, RNA is subject to a number of covalent modifications that can impact its function and post-transcriptionally modified nucleotides have indeed been detected on eukaryotic RNAs. Of these, the N⁶-methyladenosine (m⁶A) modification is the most common, with an average of ˜3 m⁶A addition sites per mRNA and with ˜25% of all cellular mRNAs generally containing multiple m⁶A residues. The importance of m⁶A is underlined by the fact that this modification is evolutionarily conserved from fungi to plants and animals, and that global inhibition of m⁶A addition is embryonic lethal in plants, insects and mammals.

The post-transcriptional addition of m⁶A to mRNAs occurs predominantly in the nucleus and is mediated by a heterotrimeric protein complex consisting of the two methyltransferase-like (METTL) enzymes METTL3 and METTL14 and their co-factor Wilms tumor 1-associated protein (WTAP). This complex specifically methylates A residues in the consensus sequence (G/A/U)(G>A) m⁶AC (U/C/A). In addition to these m⁶A “writers”, mammals also encode two RNA demethylases or “erasers” called ALKBH5 (α-ketoglutamarate-dependent dioxygenase homologue 5) and FTO (fat mass and obesity associated), which are found predominantly in the nucleus or cytoplasm, respectively. Finally, the function of m⁶A residues on RNAs is thought to be primarily mediated by three related cytoplasmic “reader” proteins called YTH-domain containing family 1 (YTHDF1), YTHDF2 and YTHDF3. The three YTHDF proteins all contain a conserved carboxy-terminal YTH domain that binds m⁶A and a more variable amino-terminal effector domain of unclear function.

Although m⁶A editing of viral mRNAs was first reported 40 years ago, the role of this post-transcriptional modification is only beginning to be elucidated. Here, in part, the inventors have discovered that substitution of m⁶A sequences or m⁶A-deficient forms within a UTR region of a RNA transcript including an indicator gene will significantly affect its expression. Furthermore, the inventors observe that viruses such as influenza A encode numerous m⁶A sequences within viral open reading frames that may affect viral expression levels. This effect, which was observed in several cell types, was equivalent at both the protein and RNA level, suggesting that m⁶A sequences stabilize edited RNAs. Moreover, this effect was not specific for m⁶A sequences of viral origin as m⁶A sequences derived from human RNAs also exerted a similar positive effect on RNA and protein expression. Furthermore, the inventors were able to phenocopy the observed enhancement in RNA and protein expression induced by UTR m⁶A sequences by recruiting YTHDF proteins to the UTR of an indicator gene by fusion to an RNA-binding polypeptide recognition sequence derived from a protein-RNA tethering system, suggesting, without being limited by theory, that m⁶A sequences exert their effect by recruiting YTHDF proteins.

The inventors also observed that several of the m⁶A sequences mapped to the areas of a viral genome required for viral replication. Given their evidence that m⁶A sequences primarily act to recruit YTHDF proteins, they asked if overexpression or knockdown of YTHDF proteins would induce the predicted up or down regulation of viral replication and gene expression. The inventors did indeed observe a striking increase in viral replication when a YTHDF protein was overexpressed and a marked decline in viral replication in cells in which the a YTHDF gene had been inactivated by DNA editing. Together, these data therefore reveal that m⁶A sequences of either viral or cellular origin enhance gene expression in cis and finally demonstrate that the level of expression of cellular YTHDF proteins impacts the level of viral gene expression and replication in several types of cells in culture, as indeed predicted if the role of m⁶A sequences is to recruit YTHDF proteins to the RNA.

In one aspect of the present invention, polynucleotides are provided. As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand).

Regarding polynucleotide sequences, the terms “percent identity” and “% identity” and “% sequence identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent sequence identity for a polynucleotide may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 2, at least 3, at least 10, at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a polynucleotide sequence having at least 50% sequence identity to the particular polynucleotide over a certain length of one of the polynucleotide sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polynucleotides may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Isolated polynucleotides homologous to the polynucleotides described herein are also provided. Those of skill in the art also understand the degeneracy of the genetic code and that a variety of polynucleotides can encode the same polypeptide. In some embodiments, the polynucleotides may be codon-optimized for expression in a particular cell. While particular polynucleotide sequences which are found in viruses and humans are disclosed herein any polynucleotide sequences may be used which encode a desired form of the substituted polypeptides described herein. Thus non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The polynucleotides may include at least one m⁶A sequence. The N⁶-methyladenosine (m⁶A) modification of RNA is one of the most common post-transcriptional modifications detected in RNAs. As used herein, a “m⁶A sequence” is a RNA sequence that (1) includes the consensus sequence (G/A/U)(G>A) m⁶AC (U/C/A) and (2) is methylated within the central adenosine nucleotide of the consensus sequence in a cell. Both requirements are needed because, as known in the art, not every RNA sequence including the consensus sequence will necessarily be methylated in a cell. In other words, the consensus sequence is necessary but not sufficient to being a “m⁶A sequence.” The methylation of the consensus sequence may be detected by determining, for example and without limitation, whether the m⁶A sequence is bound sufficiently by an m⁶A specific antibody and/or a YTHDF polypeptide to indicate that the central adenosine of the consensus sequence is methylated. As used herein, a m⁶A sequence may also be a DNA sequence encoding such an RNA sequence. In some embodiments, the m⁶A sequence may include the central adenosine that is methylated in a cell and have 40%, 60%, or 80% sequence identity with the remaining nucleotides in the (G/A/U)(G>A) m⁶AC (U/C/A) consensus sequence. In some embodiments, the m⁶A sequence may require additional surrounding sequences to allow for methylation and concomitant increased gene expression, protein production and viral replication when used recombinantly. These surrounding sequences may be 5′ or 3′ to the m⁶A consensus sequence and may be at least 5, 6, 8, 10, 15, 20, 25, 30, 35, 40 or more nucleotides in length. In some embodiments, the m⁶A sequence comprises any one of SEQ ID NOS: 16-33. In some embodiments, the polynucleotide may include at least 1, 2, 3, 4, 5, 6, 7, 8, 9, 10, 11, 12, 13, 14, 15, 16, 17, 18, 19, or more m⁶A sequences. Within the polynucleotide sequence, the m⁶A sequences may be separated by at least 5, 10, 15, 20, 25, 30, 35, 40, 45, 50, 55, 60, or more bases.

The m⁶A sequences described herein may be either “engineered m⁶A sequence(s)” or “native m⁶A sequence(s).” As used herein, “engineered m⁶A sequences” are m⁶A sequences that are not found naturally in a given polynucleotide but rather are introduced into the polynucleotide using laboratory methods. “Native m⁶A sequences” are m⁶A sequences that are found naturally in a given polynucleotide.

In some embodiments, the polynucleotides may encode a heterologous polypeptide. In some embodiments it is envisioned that at least 1, 2, 3, 4, 5, 6, 7, 8, 9, 10, 11, 12, 13, 14, 15, 16, 17, 18, 19, or more engineered m⁶A sequences are incorporated into the polynucleotide encoding the heterologous polypeptide that may change or not change the amino acid sequence of the heterologous polypeptide. For example, similar to how polynucleotides are often “codon-optimized” for expression in a particular cell, it is contemplated that one or more engineered m⁶A sequences may be incorporated into a polynucleotide encoding a heterologous polypeptide which do not alter the amino acid sequence of the polypeptide but increase the expression of the heterologous polypeptide in a cell.

In some embodiments, the polynucleotides may encode a regulatory RNA. Regulatory RNAs may include, without limitation, antisense RNAs, CRISPR RNAs, guide RNAs, long noncoding RNAs, microRNAs, siRNAs. In some embodiments it is envisioned that at least 1, 2, 3, 4, 5, 6, 7, 8, 9, 10, 11, 12, 13, 14, 15, 16, 17, 18, 19, or more engineered m⁶A sequences are incorporated into the polynucleotide encoding the regulatory RNA. Like with polynucleotides encoding polypeptides, it is contemplated that engineered m⁶A sequences may be incorporated into a polynucleotide encoding a regulatory RNA which increase the expression of the regulatory RNA in a cell.

In some embodiments, the polynucleotides may encode a UTR sequence. A “UTR sequence” is a polynucleotide sequence that when expressed in a cell may, when DNA, be transcribed but, when RNA, is not typically translated. The UTR sequence may be a 3′ UTR sequence or a 5′ UTR sequence. The UTR sequence forms part of a RNA transcript that is not translated (i.e., outside the coding region for the polypeptide). In some embodiments, the UTR sequence may comprise any one of SEQ ID NOS: 1-3, 7-15, variants of SEQ ID NOS: 1-3, 7-15 having at least 60%, 65%, 70%, 75%, 80%, 85%, 90%, 95%, 98%, or 99% sequence identity to SEQ ID NOS: 1-3, 7-15, or fragments of SEQ ID NOS: 1-3, 7-15.

In some embodiments, the polynucleotides may be included within a virus. Suitable viruses are described further below.

The polynucleotides or polypeptides provided herein may be prepared by methods available to those of skill in the art. Generally, the nomenclature used herein and the laboratory procedures utilized in the present invention include molecular, biochemical, microbiological and recombinant DNA techniques that are well known and commonly employed in the art. Standard techniques available to those skilled in the art may be used for cloning, DNA and RNA isolation, amplification and purification. Such techniques are thoroughly explained in the literature.

In a further aspect of the present invention, constructs are provided. Notably each of the constructs claimed are recombinant molecules and as such do not occur in nature. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic.

The constructs of the present invention may include a promoter operably connected to anyone of the polynucleotides described herein. Such embodiments may further include a polyA site. Optionally, the construct may include in the 5′ to 3′ direction of at least one strand of the construct the promoter, the polynucleotide including at least one m⁶A site, and the polyA site.

Alternatively, the constructs of the present invention may also include an insert site, and any one of the polynucleotides encoding UTR sequence described herein. The UTR sequence may be either 5′ or 3′ to the insert site. Such embodiments may optionally further include a polyA site. The construct may include in the 5′ to 3′ direction of at least one strand of the construct the insert site, the UTR sequence, and the polyA site. In some embodiments, the construct further includes a promoter operably connected to the insert site.

As used herein, an “insert site” is a polynucleotide sequence that allows the incorporation of another polynucleotide of interest. Exemplary insert sites may include, without limitation, polynucleotides including sequences recognized by one or more restriction enzymes (i.e., multicloning site (MCS)), polynucleotides including sequences recognized by site-specific recombination systems such as the λ phage recombination system (i.e., Gateway Cloning technology), the FLP/FRT system, and the Cre/lox system or polynucleotides including sequences that may be targeted by the CRISPR/Cas system. The insert site may comprise a heterologous coding sequence encoding a heterologous polypeptide or may include any one of the polynucleotides encoding a heterologous polypeptide or a regulatory RNA described herein.

As used herein, a “polyA site” or “polyA sequence” is a polynucleotide sequence that includes 5 or more adenosine bases or a DNA sequence that encodes such a string of adenosine bases in at least one strand or may be polynucleotide sequence that signals the addition of a polyA tail to a RNA transcript. Common polyA sequences are known in the art and may include, without limitation, polyA sequences derived from the SV40 virus, from HIV-1 or from the human or rat insulin genomic gene, the human growth hormone gene or any other mammalian mRNA encoding gene. Synthetic poly(A) addition sequences, generally consisting of the sequence 5′-AAUAA-3 linked to a 3′ G/U rich sequence, can also be used.

As used herein, the terms “promoter,” “promoter region,” or “promoter sequence” refer generally to transcriptional regulatory regions of a gene or regulatory RNA (i.e., promoters, enhancers, or both), which may be found at the 5′ or 3′ side of the gene or regulatory RNA, or within the coding region of a gene or regulatory RNA, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

As used herein, a polynucleotide is “operably connected” or “operably linked” when it is placed into a functional relationship with a second polynucleotide sequence. For instance, a promoter is operably linked to an insert site or heterologous coding sequence within the insert site if the promoter is connected to the coding sequence or insert site such that it may effect transcription of the coding sequence. In various embodiments, the polynucleotides may be operably linked to at least 1, at least 2, at least 3, at least 4, at least 5, or at least 10 promoters.

Promoters useful in the practice of the present invention include, but are not limited to, constitutive, inducible, temporally-regulated, developmentally regulated, chemically regulated, tissue-preferred and tissue-specific promoters. Suitable promoters for expression in plants include, without limitation, the 35S promoter of the cauliflower mosaic virus, ubiquitine, tCUP cryptic constitutive promoter, the Rsyn7 promoter, pathogen-inducible promoters, the maize In2-2 promoter, the tobacco PR-1a promoter, glucocorticoid-inducible promoters, estrogen-inducible promoters and tetracycline-inducible and tetracycline-repressible promoters. Other promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. Those of skill in the art are familiar with a wide variety of additional promoters for use in various cell types.

The constructs of the present invention may include a heterologous coding sequence encoding a heterologous polypeptide within the insert site. The heterologous coding sequence thus may be 3′ or 5′ to the UTR sequence. In some embodiments, the expression of the constructs of the present invention in a cell produces a transcript including the heterologous coding sequence and the UTR sequence. A “heterologous coding sequence” is a region of a construct that is an identifiable segment (or segments) that is not found in association with the larger construct in nature. When the heterologous coding region encodes a gene or a portion of a gene, the gene may be flanked by DNA that does not flank the genetic DNA in the genome of the source organism. In another example, a heterologous coding region is a construct where the coding sequence itself is not found in nature.

As used herein, a “heterologous polypeptide” “polypeptide” or “protein” or “peptide” may be used interchangeably to refer to a polymer of amino acids. A “polypeptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

In some embodiments, the heterologous polypeptide may be a therapeutic polypeptide, industrial enzyme or other useful protein product. Exemplary therapeutic polypeptides are summarized in, for example Leader et al., Nature Review—Drug Discovery 7:21-39 (2008). Therapeutic polypeptides include but are not limited to enzymes, antibodies, hormones, cytokines, ligands, protein antigens, competitive inhibitors and can be naturally occurring or engineered polypeptides. The therapeutic polypeptides may include, without limitation, Insulin, Pramlintide acetate, Growth hormone (GH), somatotropin, Mecasermin, Mecasermin rinfabate, Factor VIII, Factor IX, Antithrombin III (AT-III), Protein C, beta-Gluco-cerebrosidase, Alglucosidase-alpha, Laronidase, Idursulphase, Galsulphase, Agalsidase-beta, alpha-1-Proteinase inhibitor, Lactase, Pancreatic enzymes (lipase, amylase, protease), Adenosine deaminase, immunoglobulins, Human albumin, Erythropoietin, Darbepoetin-alpha, Filgrastim, Pegfilgrastim, Sargramostim, Oprelvekin, Human follicle-stimulating hormone (FSH), Human chorionic gonadotropin (HCG), Lutropin-alpha, Type I alpha-interferon, Interferon-alpha2a, Interferon-alpha2b, Interferon-alphan3, Interferon-beta1a, Interferon-beta1b, Interferon-gammalb, Aldesleukin, Alteplase, Reteplase, Tenecteplase, Urokinase, Factor VIIa, Drotrecogin-alpha, Salmon calcitonin, Teriparatide, Exenatide, Octreotide, Dibotermin-alpha, Recombinant human bone morphogenic protein 7 (rhBMP7), Histrelin acetate, Palifermin, Becaplermin, Trypsin, Nesiritide, Botulinumtoxin type A, Botulinum toxin type B, Collagenase, Human deoxy-ribonuclease I, dornase-alpha, Hyaluronidase (bovine, ovine), Hyaluronidase (recombinant human, Papain, L-Asparaginase, Rasburicase, Lepirudin, Bivalirudin, Streptokinase, Anistreplase, Bevacizumab, Cetuximab, Panitumumab, Alemtuzumab, Rituximab, Trastuzumab, Abatacept, Anakinra, Adalimumab, Etanercept, Infliximab, Alefacept, Efalizumab, Natalizumab, Eculizumab, Antithymocyte globulin (rabbit), Basiliximab, Daclizumab, Muromonab-CD3, Omalizumab, Palivizumab, Enfuvirtide, Abciximab, Pegvisomant, Crotalidae polyvalent immune Fab (ovine), Digoxin immune serum Fab (ovine), Ranibizumab, Denileukin diftitox, Ibritumomab tiuxetan, Gemtuzumab ozogamicin, Tositumomab, Hepatitis B surface antigen (HBsAg), HPV vaccine, OspA, Anti-Rhesus (Rh) immunoglobulin G98 Rhophylac, Recombinant purified protein derivative (DPPD), Glucagon, Growth hormone releasing hormone (GHRH), Secretin, Thyroid stimulating hormone (TSH), thyrotropin, Capromab pendetide, Satumomab pendetide, Arcitumomab, Nofetumomab, Apcitide, Imciromab pentetate, Technetium fanolesomab, HIV antigens, and Hepatitis C antigens.

The heterologous polypeptide may also be a Cas protein including, without limitation, Cas9. The Cas9 proteins may be derived from any bacterial genome including, without limitation, Cas9 proteins derived from Streptococcus pyogenes and Staphylococcus aureus.

Vectors including any of the polynucleotides or constructs described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Another type of vector is a viral vector (e.g., replication defective retroviruses, herpes simplex virus, lentiviruses, adenoviruses and adeno-associated viruses), where additional polynucleotide segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Yeast and bacterial artificial chromosomes are also included as vectors.

Cells including any of the polynucleotides, constructs, or vectors described herein are provided. Suitable “cells” include eukaryotic cells. Suitable eukaryotic cells include, without limitation, plant cells, fungal cells, and animal cells such as cells from popular model organisms including, but not limited to, Saccharomyces cerevisiae, Caenorhabditis elegans, Drosophila melanogaster, Danio rerio, Mus musculus, and Rattus norvegicus. In some embodiments, the cell may be a mammalian cell, a chicken cell, or an insect cell. Suitable mammalian cells include, without limitation, a mouse cell, a rat cell, a hamster cell, or a human cell. Suitable chicken cells include, without limitation, primary chicken cells such as chick embryo fibroblasts, chicken cell lines, or cells within an embryonated chicken egg. In some embodiments, the cell is a mammalian cell such as, without limitation, a mouse cell, a rat cell, a hamster cell, or a human cell. The cell may be a cell line typically used to recombinantly produce polypeptides including, without limitation, insect cell lines infected by baculovirus, yeast cell lines, and mammalian cell lines such A549 cells, CHO cells, HEK293 cells, HEK293T cells, HeLa cells, NS0 cells, Sp2/0 cells, COS cells, BK cells, MDCK cells, a T cell such as a CD4 T cell, and Vero cells. Cell lines typically used for protein production are described elsewhere, for example, in Khan et al., Advanced Pharmaceutical Bulletin 3(2): 257-263 (2013). In some embodiments, the cell may be a cell line used to produce viruses including, without limitation, insect cells, chicken cells, HEK 293 cells, HEK 293T cells, A549 cells and Vero cells.

In some embodiments, the cell may overexpress a YTHDF polypeptide. As used herein, “overexpressing” or “expressing” a polynucleotide or polypeptide in a cell refers to transcribing or translating a polynucleotide or polypeptide that has been introduced into the cell using laboratory methods. For example, the polypeptide may be expressed from a polynucleotide present in a vector for propagating the polynucleotide or the polypeptide may be expressed from a polynucleotide that is integrated into the genome of the cell. Overexpressing also includes increasing production of the native polypeptide by altering expression of the native polypeptide. Overexpression of the native polypeptide may be accomplished by any means available to those skilled in the art, including adding enhancers, altering the promoter, supplying a trans activating factor or any other means.

The function of m⁶A residues on RNAs is thought to be primarily mediated by three related cytoplasmic “reader” protein families called YTH-domain containing family 1 YTHDF1, YTHDF2 and YTHDF3. As used herein, a “YTHDF polypeptide” may refer to YTHDF1, YTHDF2 or YTHDF3 polypeptides from any eukaryote. Suitably, the YTHDF polypeptide could be from the organism from which the cell is derived or within. In some embodiments, the YTHDF polypeptide is the polypeptide of SEQ ID NO: 4, SEQ ID NO: 5, SEQ ID NO: 6, or a polypeptide having at least 60%, 70%, 75%, 80%, 85%, 90%, 95%, or 98% sequence identity to SEQ ID NO: 4, SEQ ID NO: 5, or SEQ ID NO: 6. In mammals, YTHDF polypeptides have been well-conserved throughout evolution showing on the order of greater than 90% sequence identity. See, e.g., SEQ ID NOs: 34-40.

The polypeptides contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine) or other enzymatic attachments are also encompassed.

The polypeptides disclosed herein may include “mutant” polypeptides, variants, and derivatives thereof. As used herein the term “wild-type” is a term of the art understood by skilled persons and means the typical form of an organism, strain, gene or characteristic as it occurs in nature as distinguished from mutant or variant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a YTHDF mutant or variant polypeptide may have one or more insertions, deletions, or substitution of at least one amino acid residue relative to the YTHDF “wild-type” polypeptide. The polypeptide sequences of the “wild-type” YTHDF1, YTHDF2, and YTHDF polypeptides from humans is presented as SEQ ID NO: 4, SEQ ID NO: 5, and SEQ ID NO: 6, respectively. These sequences may be used as reference sequences.

SEQ ID NOs: 34-40 are the YTHDF2 proteins from Goat, Cat, Zebu, Gray Mouse, Beaver, Rat and a consensus sequence. Based on alignment of these sequences it becomes immediately apparent to a person of ordinary skill in the art that various amino acid residues may be altered (i.e. substituted, deleted, etc.) in, for example human YTHDF2 (SEQ ID NO: 5), without substantially affecting the activity of the polypeptide. For example, a person of ordinary skill in the art would appreciate that substitutions in a reference YTHDF2 (i.e., human) could be based on alternative amino acid residues that occur at the corresponding position in other YTHDF2 polypeptides from other species. For example, the human YTHDF2 polypeptide has a asparagine amino acid residue at position 174 while some of the other polypeptides have a serine amino acid at this position in the alignment. Thus, one exemplary modification that is apparent from the sequence alignment of these sequences is a N174S in the human YTHDF2 polypeptide (SEQ ID NO: 5). Similar modifications could be made at each position of the sequence alignments of the various YTHDF sequences provided herein. Additionally, a person of ordinary skill in the art, could easily align other YTHDF2 polypeptides with the polypeptide sequences shown here to determine what additional variants could be made to YTHDF2 polypeptides.

The polypeptides provided herein may be full-length polypeptides or may be fragments of the full-length polypeptide. As used herein, a “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide. A fragment of a YTHDF polypeptide may comprise or consist essentially of a contiguous portion of an amino acid sequence of the full-length YTHDF polypeptide (SEQ ID NOS: 4, 5, or 6). A fragment may include an N-terminal truncation, a C-terminal truncation, or both truncations relative to the full-length YTHDF polypeptide. Preferably, a fragment of an YTHDF polypeptide includes amino acid residues required for the m⁶A reader function.

A “deletion” in a polypeptide refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in a polypeptide refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant of a YTHDF polypeptide may have N-terminal insertions, C-terminal insertions, internal insertions, or any combination of N-terminal insertions, C-terminal insertions, and internal insertions.

Regarding polypeptides, the phrases “percent identity,” “% identity,” and “% sequence identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases. As described herein, variants, mutants, or fragments (e.g., a YTHDF polypeptide variant, mutant, or fragment thereof) may have 99%, 98%, 97%, 96%, 95%, 94%, 93%, 92%, 91%, 90%, 80%, 70%, 60%, or 50% amino acid sequence identity relative to a reference molecule (e.g., relative to the YTHDF full-length polypeptide (SEQ ID NO: 2)).

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The amino acid sequences of the polypeptide variants, mutants, or derivatives as contemplated herein may include conservative amino acid substitutions relative to a reference amino acid sequence. For example, a variant, mutant, or derivative polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

Methods for producing a heterologous polypeptide in a cell are provided. The methods may include introducing any of the polynucleotides, constructs, or vectors described herein into the cell. Suitably, the polynucleotides, constructs, and vectors include a heterologous coding sequence encoding a heterologous polypeptide.

As used herein, “introducing” describes a process by which exogenous polynucleotides (e.g., DNA or RNA) or protein are introduced into a recipient cell. Methods of introducing polynucleotides and proteins into a cell are known in the art and may include, without limitation, microinjection, transformation, and transfection methods. Transformation or transfection may occur under natural or artificial conditions according to various methods well known in the art, and may rely on any known method for the insertion of foreign nucleic acid sequences into a host cell. The method for transformation or transfection is selected based on the type of host cell being transformed and may include, but is not limited to, bacteriophage or viral infection, electroporation, heat shock, lipofection, and particle bombardment. Microinjection of polynucleotides and/or proteins may also be used to introduce polynucleotides and/or proteins into cells.

The polynucleotides, constructs, and vectors of the present invention may also be formulated for delivery into a human subject. For example, it is envisioned that mRNAs produced using the constructs described herein either in cells or in an in vitro transcription system may be delivered to human cells using mRNA delivery platforms like those developed by, for example, Moderna Therapeutics.

Conventional viral and non-viral based gene transfer methods can be used to introduce polynucleotides into cells or target tissues. Non-viral vector delivery systems include DNA plasmids, RNA, naked nucleic acid, and nucleic acid complexed with a delivery vehicle, such as a liposome. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

In some embodiments, the methods may further include expressing a YTHDF polypeptide in the cell.

The methods may also further include additional steps used in producing polypeptides recombinantly. For example, the methods may include purifying the heterologous polypeptide from the cell. The term “purifying” is used to refer to the process of ensuring that the heterologous polypeptide is substantially or essentially free from cellular components and other impurities. Purification of polypeptides is typically performed using molecular biology and analytical chemistry techniques such as polyacrylamide gel electrophoresis or high performance liquid chromatography. Methods of purifying protein are well known to those skilled in the art. A “purified” heterologous polypeptide means that the heterologous polypeptide is at least 85% pure, more preferably at least 95% pure, and most preferably at least 99% pure.

The methods may also include the step of formulating the heterologous polypeptide into a therapeutic for administration to a subject.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, mice, chickens, amphibians, reptiles, and the like. Preferably, the subject is a human patient. More preferably, the subject is a human patient in need of a heterologous polypeptide or a vaccine.

Fusion proteins, constructs encoding these fusion proteins and cells including these constructs or capable of expressing these fusion proteins are also provided. The fusion protein may include a YTHDF polypeptide and a RNA-binding polypeptide. The terms “fusion protein” and “fusion polypeptide” may be used to refer to a single polypeptide comprising two functional segments, e.g., a YTHDF polypeptide segment and a RNA-binding polypeptide. The fusion proteins may be any size, and the single polypeptide of the fusion protein may exist in a multimeric form in its functional state, e.g., by cysteine disulfide connection of two monomers of the single polypeptide. A polypeptide segment may be a synthetic polypeptide or a naturally occurring polypeptide. Such polypeptides may be a portion of a polypeptide or may comprise one or more mutations. The two polypeptide segments of the fusion proteins can be linked directly or indirectly. For instance, the two segments may be linked directly through, e.g., a peptide bond or chemical cross-linking, or indirectly, through, e.g., a linker segment or linker polypeptide. The peptide linker may be any length and may include traditional or non-traditional amino acids. For example, the peptide linker may be 1-100 amino acids long, suitably it is 5, 10, 15, 20, 25 or more amino acids long such that the YTHDF portion of the fusion polypeptide can mediate its m⁶A reader function and the RNA-binding polypeptide can bind its recognition requence.

A “RNA-binding polypeptide” may be any of the RNA-binding polypeptides commonly employed in protein-RNA tethering systems. Protein-RNA tethering systems have been summarized in, for example, Coller and Wickens, Methods of Enzymology 429:299-(2007). In choosing which RNA-binding polypeptide to use as the tether, it is necessary to consider the affinity and specificity for the RNA-binding polypeptide recognition sequence, subcellular localization, and impact of the tether on the activity of the YTHDF polypeptide. The most common RNA-binding polypeptide, and the RNA-binding polypeptide used in the Examples, is the bacteriophage MS2 coat protein. However, the iron response element binding protein (IRP), a derivative of bacteriophage λN-protein, and the spliceosomal U1A protein have been used successfully. Therefore, in some embodiments, the RNA-binding polypeptide may include, without limitation, a MS2 polypeptide, a lambda N polypeptide, an iron response element binding polypeptide, or U1A polypeptide.

The constructs of the present invention may also include (i) a heterologous coding sequence encoding a heterologous polypeptide, and (ii) a UTR sequence including at least one RNA-binding polypeptide recognition sequence. The UTR sequence may be located either 5′ or 3′ to the heterologous coding sequence. In one embodiment, the UTR sequence is 3′ to the heterologous coding sequence. The RNA-binding polypeptide recognition sequence is a polynucleotide sequence recognized and bound by the RNA-binding polypeptide. The recognition sequences often form a stem loop structure. Suitable RNA-binding polypeptide recognition sequences for the MS2, lambda N, iron response element binding, and U1A RNA-binding polypeptides are known in the art. In some embodiments, the UTR sequence may include at least 1, 2, 3, 4, 5, 6, 7, 8, 9, 10, 11, 12, 13, 14, 15, 16, 17, 18, 19, or more RNA-binding polypeptide recognition sequences. Within the UTR sequence, the RNA-binding polypeptide recognition sequences may be separated by at least 5, 10, 15, 20, 25, 30, 35, 40, 45, 50, 55, 60, or more bases. Cells including any of the constructs including RNA-binding polypeptide recognition sequences are also provided.

Methods for producing a heterologous polypeptide in a cell including (a) introducing or expressing any of the fusion proteins described herein or the constructs encoding such fusion proteins into the cell, and (b) introducing or expressing any of the constructs including RNA-binding polypeptide recognition sequences described herein. Such methods may also further include additional steps used in producing therapeutic polypeptides recombinantly. For example, the methods may include purifying the heterologous polypeptide from the cell. Such methods may also include the step of formulating the heterologous polypeptide into a therapeutic for administration to a subject as described more fully above.

Cells engineered to overexpress a YTHDF polypeptide are provided. Optionally, the YTHDF overexpressing cells may include a virus comprising at least one m⁶A sequence. As used herein, a “virus” may include any virus or viral vector including at least one m⁶A sequence or any of the viruses including the polynucleotides described herein. In some embodiments, the virus may be a nucleovirus that replicates in the nucleus of a cell. In some embodiments, the virus may be a virus used to make vaccines such as, without limitation, a measles virus, a mumps virus, a rubella virus, an influenza virus, a varicella-zoster virus, a polio virus, a rotavirus, a yellow fever virus, a rabies virus, or other viruses that may be used in the production of a vaccine or for making viral stocks for use in research or other applications. An influenza virus includes, but is not limited to an influenza A, B, or C virus. In some embodiments, the virus may be a live-attenuated virus or a live virus. In some embodiments, the virus may be a virus or viral vector used in gene therapy applications such as, without limitation, a retrovirus, an adenovirus such as AAV, or a Herpes simplex virus. In some embodiments, the retrovirus may be a lentivirus. In some embodiments, the virus may include, without limitation, an Adeno Associated Virus (AAV), influenza viruses (types A-C), Human Immunodeficiency Viruses (HIV) or other viruses that may be used in the production of viral vectors (for example, for gene therapy). In some embodiments, the virus may include viruses expressed from an engineered plasmid system such as a YAC or BAC or may be native viruses.

Methods of producing a virus in a cell are provided. The methods may include (a) introducing the virus into the cell, wherein the virus comprises at least one m⁶A sequence and (b) introducing or expressing a YTHDF polypeptide in the cell. Such methods may also further include additional steps used in producing vaccines. For example, the methods may include purifying the virus from the cell. In some embodiments, the virus may be killed following purification from the cell. Such methods may also include the step of formulating the virus (whether live-attenuated or killed) into a vaccine for administration to a subject.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

The following examples are meant only to be illustrative and are not meant as limitations on the scope of the invention or of the appended claims.

### EXAMPLES

### Example 1

While the presence of multiple m⁶A editing sites on a range of viral RNAs was reported starting almost 40 years ago, how m⁶A editing affects virus replication has remained unknown. Here, we precisely map several m⁶A editing sites on the HIV-1 genome and show that these cluster in the HIV-1 3′ untranslated region (3′UTR). We demonstrate that these viral 3′UTR m⁶A sites, or analogous cellular m⁶A sites, strongly enhance mRNA expression in cis by recruiting the cellular YTHDF m⁶A “reader” proteins. As a result, inhibition of YTHDF expression was found to inhibit HIV-1 replication, while YTHDF overexpression enhanced HIV-1 replication. These data identify m⁶A editing, and the resultant recruitment of YTHDF proteins, as major positive regulators of HIV-1 mRNA expression.

Like proteins and DNA, RNA is subject to a number of covalent modifications that can impact its function and post-transcriptionally modified nucleotides have indeed been detected on eukaryotic mRNAs (Carlile et al., 2014; Dominissini et al., 2012; Dominissini et al., 2016; Meyer et al., 2012; Schwartz et al., 2014; Squires et al., 2012). Of these, the N⁶-methyladenosine (m⁶A) modification is the most common, with an average of ˜3 m⁶A addition sites per mRNA and with ˜25% of all cellular mRNAs containing generally multiple m⁶A residues (Desrosiers et al., 1975; Dominissini et al., 2012; Meyer et al., 2012). The importance of m⁶A is underlined by the fact that this modification is evolutionarily conserved from fungi to plants and animals, and that global inhibition of m⁶A addition is embryonic lethal in plants, insects and mammals (Meyer and Jaffrey, 2014; Yue et al., 2015).

The post-transcriptional addition of m⁶A to mRNAs occurs predominantly in the nucleus and is mediated by a heterotrimeric protein complex consisting of the two methyltransferase-like (METTL) enzymes METTL3 and METTL14 and their co-factor Wilms tumor 1-associated protein (WTAP) (Liu et al., 2014; Meyer and Jaffrey, 2014; Yue et al., 2015). This complex specifically methylates A residues in the consensus sequence (G/A/U)(G>A) m⁶AC (U/C/A), although only ˜15% of sites that have this consensus are actually modified and the level of modification at any given site can vary significantly. In addition to these m⁶A “writers”, mammals also encode two RNA demethylases or “erasers” called ALKBH5 (α-ketoglutamarate-dependent dioxygenase homologue 5) and FTO (fat mass and obesity associated), which are found predominantly in the nucleus or cytoplasm, respectively (Jia et al., 2011; Zheng et al., 2013). Finally, the function of m⁶A residues on mRNAs is thought to be primarily mediated by three related cytoplasmic “reader” proteins called YTH-domain containing family 1 (YTHDF1), YTHDF2 and YTHDF3 (Meyer and Jaffrey, 2014; Wang et al., 2014; Wang et al., 2015; Yue et al., 2015). The three YTHDF proteins all contain a conserved carboxy-terminal YTH domain that binds m⁶A and a more variable amino-terminal effector domain of unclear function.

While the m⁶A modification of mRNAs is therefore well established and has been suggested to modulate several aspects of RNA metabolism (Meyer and Jaffrey, 2014; Yue et al., 2015), exactly how m⁶A editing regulates mRNA function remains largely unclear. Importantly, m⁶A modifications appear to be ubiquitous on mRNAs expressed by viruses that replicate in the nucleus, including SV40, the related retroviruses avian sarcoma virus and Rous sarcoma virus (RSV), adenovirus and influenza A virus (IAV) (Dimock and Stoltzfus, 1977; Kane and Beemon, 1985; Krug et al., 1976; Lavi and Shatkin, 1975; Sommer et al., 1976). As viruses invariably rapidly evolve to maximize their replication potential, and given that it would be simple to select for viral mutants that lack consensus m⁶A modification sites, this implies that the m⁶A modification of viral mRNAs enhances viral replication by enhancing some aspect(s) of mRNA function.

Despite the fact that the identification of m⁶A on viral mRNAs dates back over 40 years, no report has shown that m⁶A affects any aspect of viral mRNA function. Here, we first precisely map m⁶A modification sites on the RNA genome of human immunodeficiency virus 1 (HIV-1) and show that different HIV-1 isolates contain from four to six m⁶A clusters at the extreme 3′ end of the viral genome, i.e., primarily in the 3′ untranslated regions (3′UTRs) of the various HIV-1 mRNAs. We further present evidence that these 3′UTR m⁶A residues enhance HIV-1 gene expression and replication by increasing the steady state level of viral mRNA expression. Finally, we show that HIV-1 is sensitive to the level of YTHDF2 expression in infected T cells, demonstrating enhanced replication when YTHDF2 was overexpressed and strongly reduced replication when the YTHDF2 gene was knocked out by DNA editing. These data demonstrate that the m⁶A modification of HIV-1 plays a key role in promoting its replication and identifies this RNA modification as a potential novel target for antiviral drug development.

### Results

### Mapping m⁶A Sites on the HIV-1 Genome

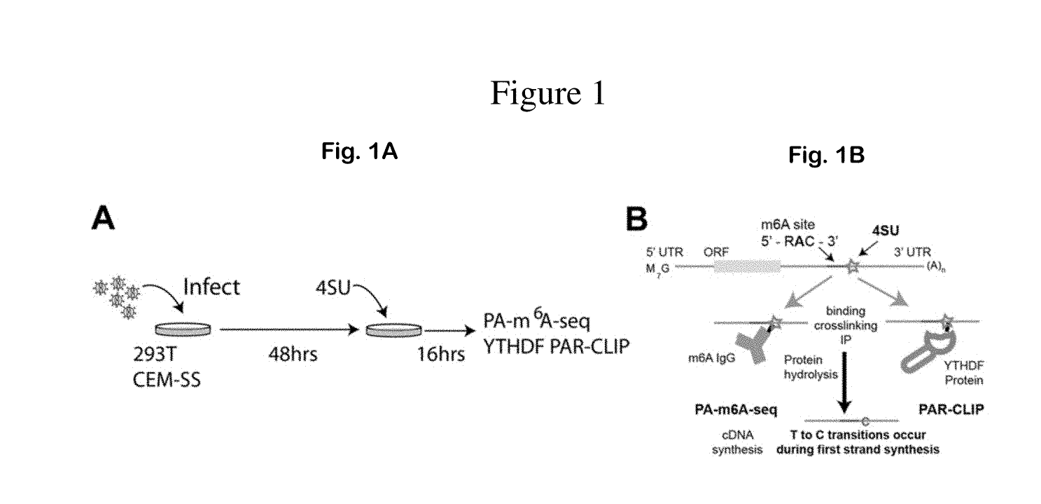

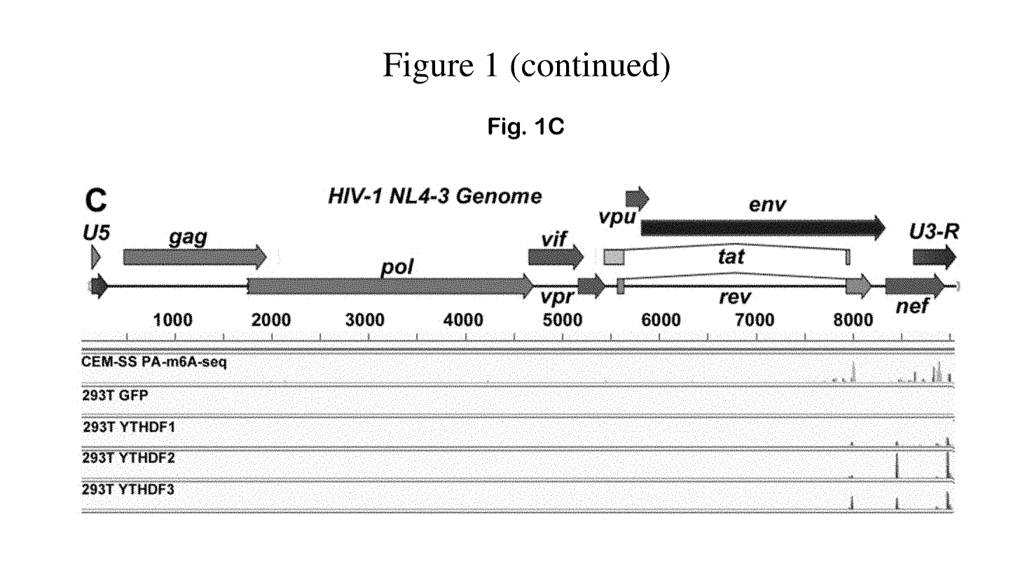

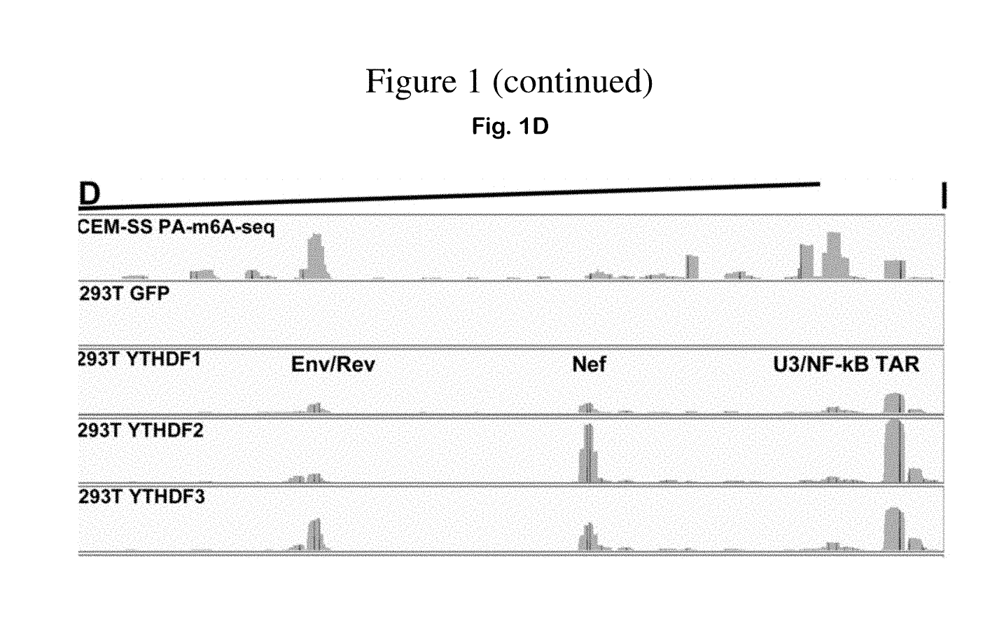

Modification of adenosines to m⁶A on viral mRNAs has been reported for a range of viruses that replicate in the nucleus; however, with the exception of RSV, where seven m⁶A addition sites were mapped using biochemical approaches (Kane and Beemon, 1985), the location of individual m⁶A residues has remained unknown. To map m⁶A modifications in HIV-1, we used the previously described photo-crosslinking-assisted m⁶A sequencing (PA-m⁶A-seq) technique (Chen et al., 2015) to identify m⁶A residues on the HIV-1 genome in infected human CD4+ CEM-SS T cells. For this experiment, we pulsed HIV-1 infected T-cells with the nucleoside 4-thiouridine (4SU), isolated total poly(A)+ RNA (FIG. 1A), bound this RNA with an m⁶A-specific antibody and crosslinked the antibody to the RNA (FIG. 1B). RNA fragments bound to the m⁶A antibody were then reverse transcribed and sequenced. We identified several ⁶A sites that were all located in the 3′ most ˜1.4 kb of the ˜9.2 kb HIV-1 RNA genome (FIG. 1C). Expansion of this region of the HIV-1 genome (FIG. 1D) reveals three major m⁶A peaks located in the overlap region between the env gene and the second coding exon of rev, in the “U3” region of the LTR, particularly in the conserved NF-κB binding sites, and finally in the “R” region of the LTR coincident with the TAR (trans-activation response) RNA hairpin, though several other minor m⁶A peaks were also visible.

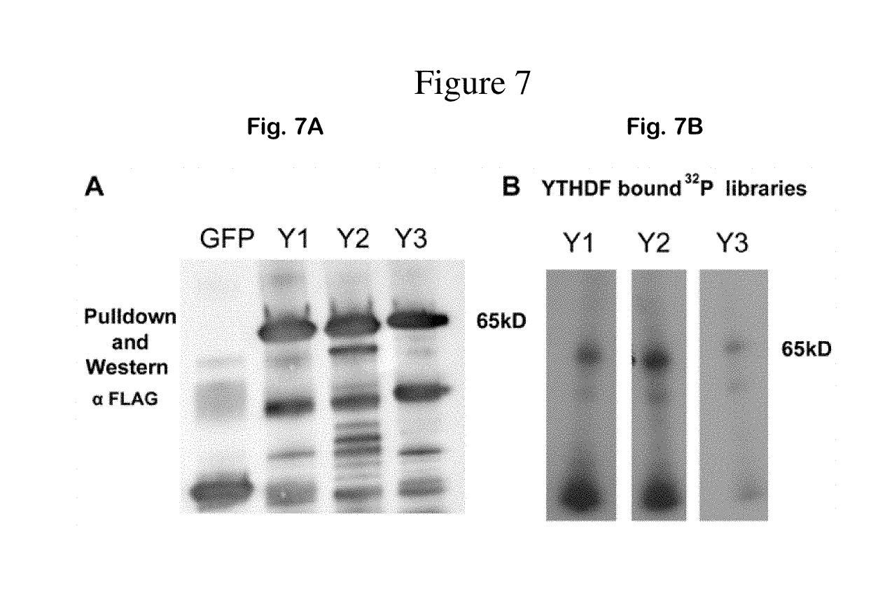

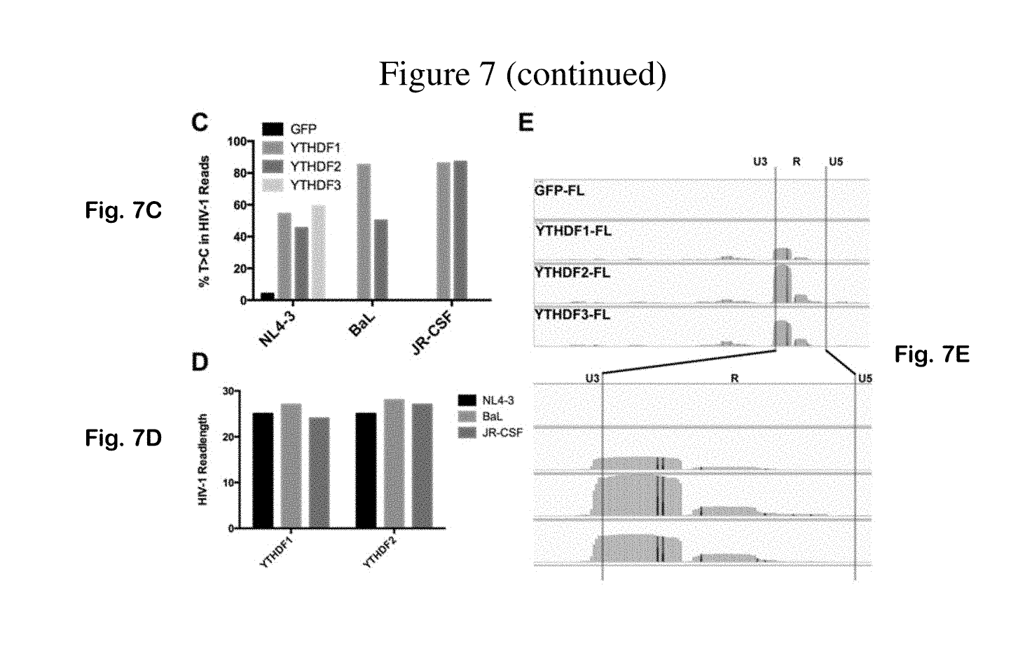

The function of m⁶A sites is primarily mediated by the cytoplasmic YTHDF proteins, though other potential nuclear or cytoplasmic m⁶A binding proteins have been reported (Meyer and Jaffrey, 2014; Meyer et al., 2015). To determine whether any of the m⁶A sites on the HIV-1 genome mapped using PA-m⁶A-seq are bound by one or more of the three YTHDF proteins in living cells, and hence likely to be functionally relevant, we generated clones of the human cell line 293T engineered to express FLAG-tagged versions of green fluorescent protein (GFP), YTHDF1, YTHDF2 or YTHDF3 (FIG. 7A). These cells were infected with a pseudotyped stock of the HIV-1 laboratory isolate NL4-3 (Adachi et al., 1986), cultured for 48 h and then incubated with 4SU for a further 16 h (FIG. 1A). At this point, the cells were subjected to photoactivatable ribonucleoside-enhanced crosslinking and immunoprecipitation (PAR-CLIP), using a monoclonal anti-FLAG antibody, followed by deep sequencing (FIG. 7B) (Hafner et al., 2010).

Analysis of recovered reads detected T to C mutations, which are characteristic of crosslinked 4SU residues that have been subjected to reverse transcription, in 45-60% of all viral reads obtained from the three FLAG-YTHDF expressing clones but in <5% of the reads obtained from the clone expressing FLAG-GFP (FIG. 7C), consistent with known ability of all three YTHDF proteins, but not GFP, to bind RNA. Alignment of reads bearing T to C mutations to the human and HIV-1 genome (reads lacking T to C mutations were discarded) revealed that the HIV-1-specific reads showed a mean length of ˜25 bp (FIG. 7D), which permitted their unequivocal assignment to the viral genome. Further analysis revealed that the HIV-1-specific reads for all three FLAG-tagged YTHDF proteins mapped to four binding clusters located in the 3′ ˜1.4 kb of the HIV-1 RNA genome, three of which coincided with the three major sites of m⁶A addition mapped by in vitro crosslinking to an m⁶A specific antibody, as described above (FIGS. 1C and D). Specifically, we noted binding clusters occupied by all three YTHDF proteins in the HIV-1 env/rev overlap; in the LTR NF-κB repeats and finally in the LTR R region. Of interest, all three YTHDF proteins also bound a site in the HIV-1 nef gene that was only detected as a minor binding site by PA-m⁶A-seq (FIG. 1D). None of the other, minor binding sites detected by PA-m⁶A-seq were bound at significant levels by any of the three YTHDF proteins. Therefore we can conclude that HIV-1 transcripts are modified by m⁶A editing at several sites located in the 3′ ˜1.4 kb of the viral RNA genome and that these m⁶A sites are bound in living cells by all three YTHDF effector proteins.

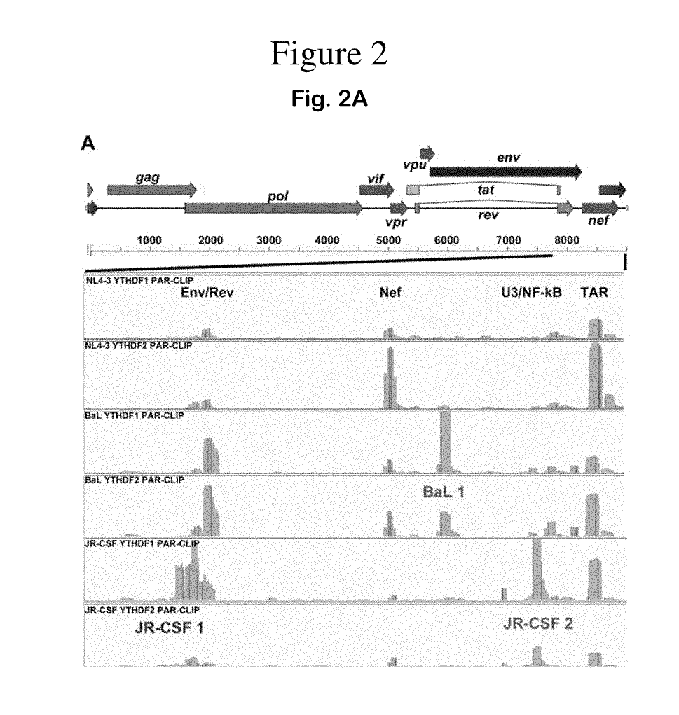

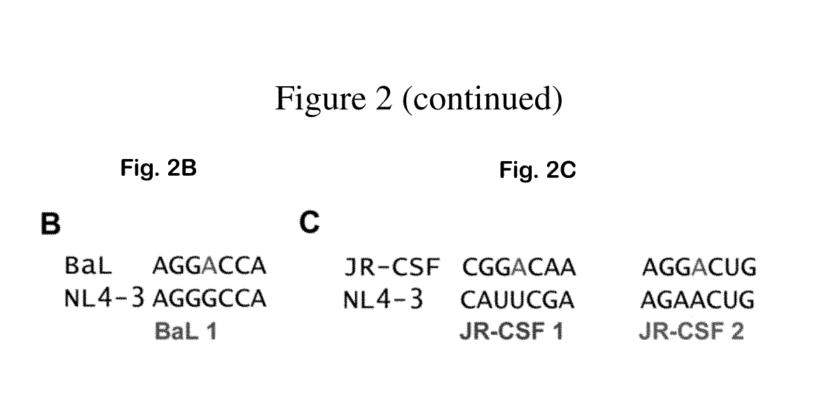

To determine if the m⁶A modification sites mapped on the NL4-3 laboratory isolate were conserved in primary HIV-1 isolates BaL and JR-CSF (Cann et al., 1990; Hwang et al., 1991), we repeated the PAR-CLIP analysis in the 293T clones expressing either FLAG-YTHDF1 or FLAG-YTHDF2 using pseudotyped stocks of BaL or JR-CSF. Analysis of the BaL isolate showed that all four clusters identified in NL4-3, in the env/rev overlap, in nef, in U3 and in TAR (FIG. 1D), were conserved in BaL and again bound by both YTHDF1 and YTHDF2 (FIG. 2A). However, we also noted a novel, intense binding site for both YTHDF proteins in the 3′ segment of nef that overlaps with the LTR U3 region (FIG. 2A, labeled BaL1). Sequence analysis revealed that this new site coincides with a consensus m⁶A modification site present in BaL (5-GGA*CC-3′) that is lacking in NL4-3 and JR-CSF (FIG. 2B).

The analysis of m⁶A editing sites in JR-CSF produced a similar result. Specifically, both YTHDF1 and YTHDF2 bound to the four m⁶A clusters previously identified in NL4-3 (FIG. 2A), while the novel m⁶A site identified in BaL was, as expected, lacking. However, in JR-CSF we identified two additional, novel m⁶A modification sites in the rev/env overlap region (JR-CSF1) and in the nef/U3 overlap region (JR-CSF2). The novel site in the rev/env overlap region again coincided with a novel “A” residue, present in JR-CSF but not NL4-3 or BaL, that forms part of an m⁶A consensus editing site (5′-GGA*CA-3′) while, in the case of the LTR U3 region target, the novel m⁶A site appeared to arise due to a change from a weaker m⁶A consensus sequence (5′-GAA*CU-3′) to a stronger consensus (5′-GGA*CU-3′) (FIG. 2C). In fact, we did detect a low level of YTHDF binding to this site in both NL4-3 and BaL, suggesting that this sequence may be subject to a low level of m⁶A modification in these viral strains (FIG. 2A). In conclusion, these data demonstrate that all four m⁶A clusters identified in the NL4-3 strain of HIV-1 are conserved in the primary isolates BaL and JR-CSF while BaL has also acquired one, and JR-CSF two, novel m⁶A sites, each lacking in the other two virus isolates. Of particular note is the fact that all YTHDF protein binding sites, including the novel ones present in BaL and JR-CSF, were located in the 3′ ˜1.4 kb of all three virus strains, with no m⁶A sites being detected in the first ˜7.8 kb of the genome (FIGS. 1D and 1E, FIG. 2A). It has recently been proposed that m⁶A sites on cellular mRNAs are concentrated in the 3′UTR (Ke et al., 2015) and indeed the m⁶A sites identified in HIV-1 would be present in the 3′ UTR of all viral mRNAs (the U3/NF-kB and TAR clusters), in the 3′ UTR of all viral mRNAs except nef mRNAs (the nef and U3/nef overlap clusters) or in the 3′UTR of the viral Gag, Gag/Pol, Vif, Vpr, Rev and Tat mRNAs (the env/rev overlap clusters).

The Introduction of m⁶A into 3′UTRs Enhances mRNA Function

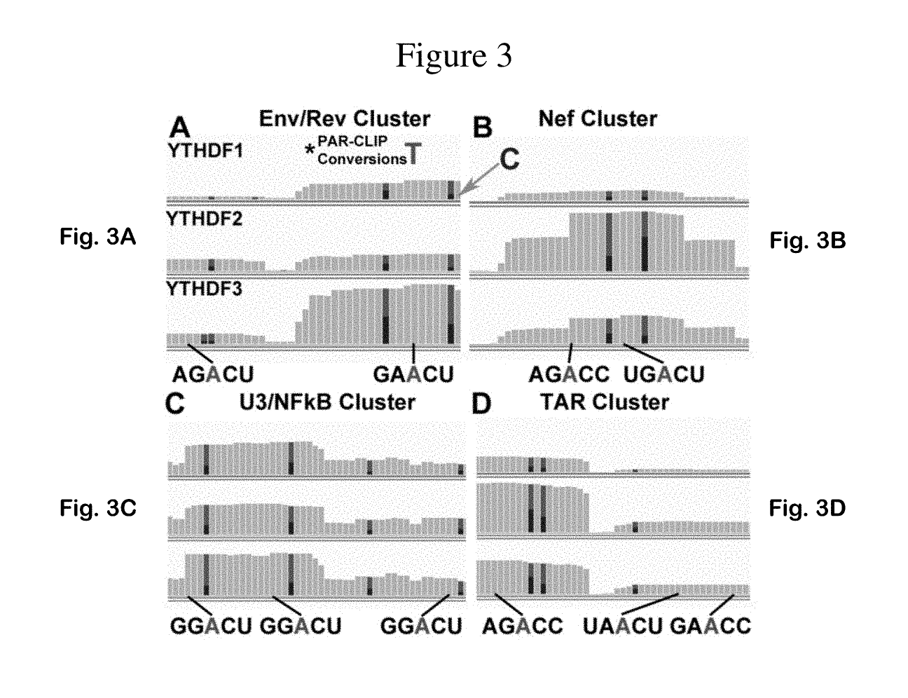

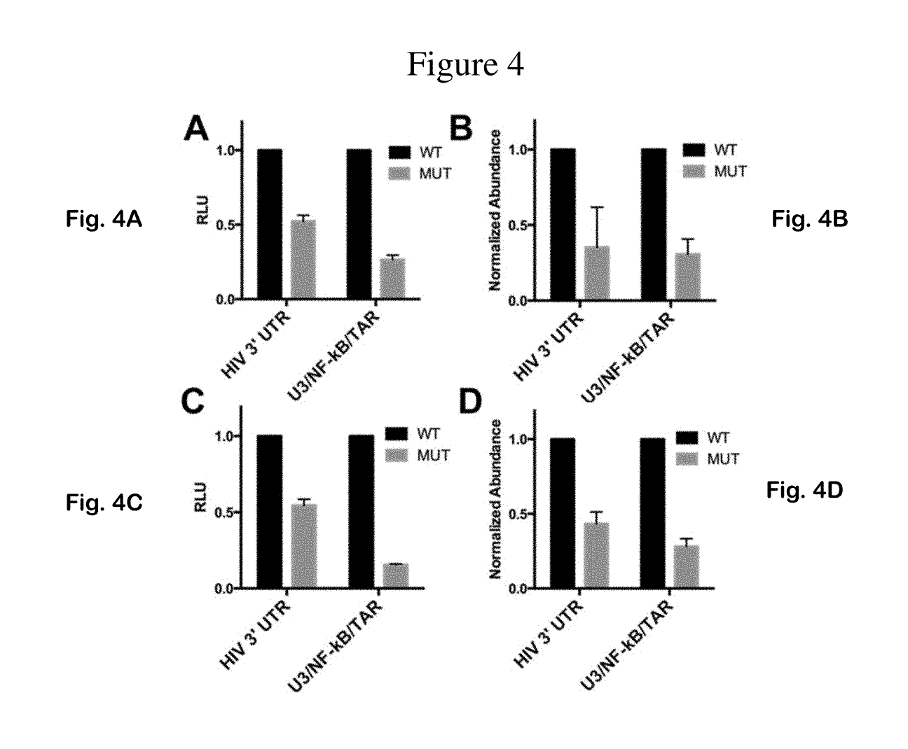

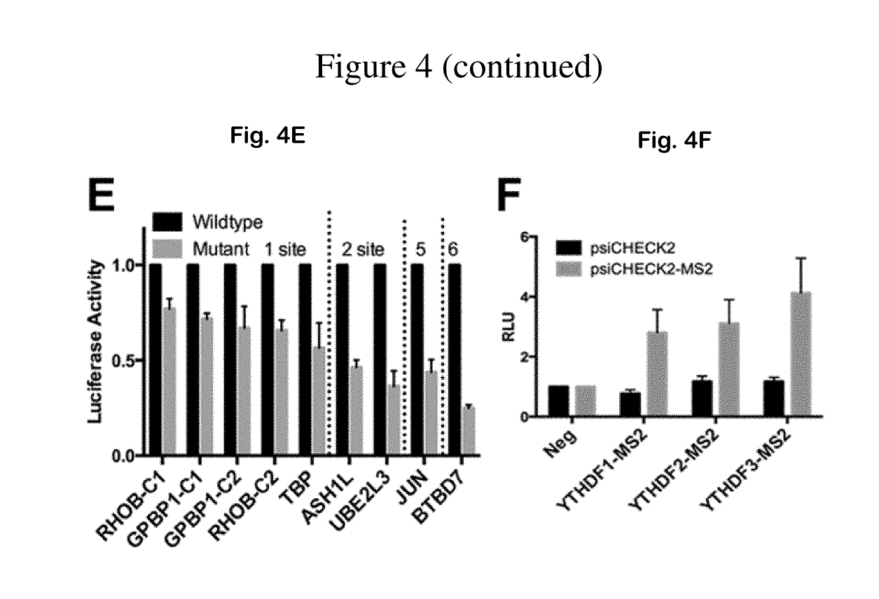

While the YTHDF and m⁶A antibody-specific binding clusters detected in the NL4-3 strain of HIV-1 are <40 nt each (FIG. 1D), they nevertheless each contain two or three potential ⁶A sites that match the minimum m⁶A consensus sequence 5′-RA*C-3′ (FIG. 3A to D). To test the effect of these clusters on mRNA function in cis, we constructed two Renilla luciferase (RLuc)-based indicator plasmids containing either the entire ˜1.4 kb region from the 3′ end of NL4-3, that encompasses all four NL4-3 m⁶A clusters, or containing just the most 3′ sequence, encompassing the U3/NF-kB and TAR clusters (FIG. 1D), in either a wildtype form or with all of the potential m⁶A modification sites listed in FIG. 3 mutated to G. These vectors contain all viral sequences required for poly(A) addition at the R/U5 junction and therefore are predicted to contain the same 3′UTRs as HIV-1 mRNAs. As shown in FIG. 4A, both indicator plasmids expressed significantly (p<0.01) higher levels of RLuc protein in transfected 293T cells when the wildtype HIV-1 sequence was utilized, compared to a similar m⁶A-deficient viral sequence. This effect was particularly notable for the indicator plasmid bearing the shorter viral 3′UTR characteristic of the viral early gene products Tat, Rev and Nef. Importantly, qRT-PCR analysis of the steady state level of the RLuc mRNA transcribed from these indicator plasmids (FIG. 4B) revealed that the viral 3′UTR m⁶A sites also exerted a very similar positive effect on the level of RLuc mRNA expression, thus suggesting that the increase in RLuc protein expression (FIG. 4A) is due to an equivalent change in the steady state level of RLuc mRNA (FIG. 4B). A closely similar positive effect of the HIV-1 3′UTR m⁶A editing sites on mRNA expression and function was also observed in the CD4+ human T-cell line CEM-SS (FIGS. 4C-D).

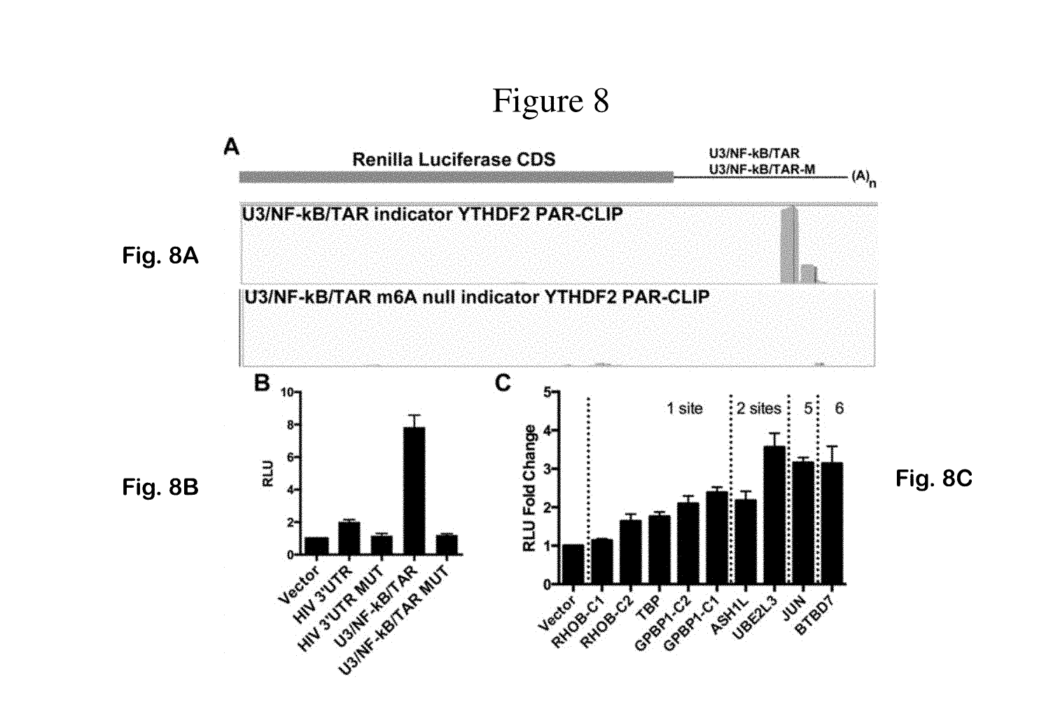

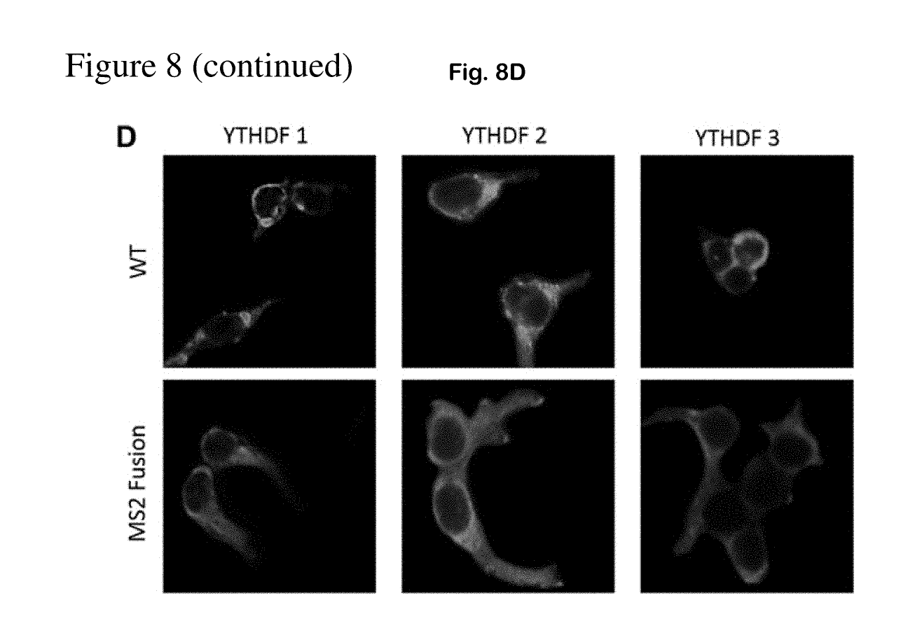

While we have mapped the m⁶A editing sites on the NL4-3 genome in the context of a virus infection (FIG. 1), it could be argued that these same m⁶A sites might be inactive in the context of an indicator plasmid. To test this possibility, we performed PAR-CLIP on 293T cells transfected with wildtype or m⁶A-deficient versions of the U3/NF-κB/TAR indicator plasmid. As shown in FIG. 8A, we observed a high level of m⁶A editing of the LTR “R” region in the wildtype context and no m⁶A editing anywhere in either the HIV-1-derived 3′UTR sequence, or the RLuc open reading frame, in the mutated indicator plasmid lacking the viral m⁶A sites. Therefore, we can conclude that the differences in gene expression observed in FIG. 4 indeed reflect the presence or absence of 3′ UTR m⁶A modifications in cis.

Previously, m⁶A editing has been proposed to either enhance or decrease mRNA stability (Dominissini et al., 2012; Wang et al., 2014) raising the possibility that m⁶A editing might exert different effects dependent on, for example, RNA sequence context. To address the generalizability of the data shown in FIGS. 4A through D, which show a clear enhancing effect of 3′UTR m⁶A editing sites of viral origin, we extended our analysis to cellular m⁶A editing sites that we had identified in human mRNAs in the course of our PAR-CLIP analyses (FIGS. 1 and 2). Specifically, we identified m⁶A editing sites bound by the three YTHDF proteins in TBP (1 cluster), RHOB (2 clusters), GPBP1 (2 clusters), ASH1L (2 clusters), UBE2L3 (2 clusters), c-jun (5 clusters) and BTBD7 (6 clusters). These cellular m⁶A editing sites were cloned into the 3′UTR of the RLuc gene, either individually or together depending on their separation in their normal sequence context, as either the wildtype sequence or with the edited A residues mutated to G. As may be observed (FIG. 4E), in all nine vectors analyzed we observed significantly lower RLuc activity when the mutant form of these human RNA sequences, lacking a consensus m⁶A addition site(s), was tested, when compared to the wildtype sequence. The observed effect was generally greater the more m⁶A residues were present (FIG. 4E).

While the data shown in FIG. 4 are presented to show a loss of RLuc activity when comparing the mutant form, lacking m⁶A sites, to the wildtype form of the inserted 3′UTR, this does not, in fact, indicate inhibition of RLuc expression by 3′UTRs lacking m⁶A sites. Rather, these data reflect the activation of RLuc expression by m⁶A containing 3′UTRs. This is more obvious when these data are normalized to a control psiCheck2 plasmid lacking any inserted 3′UTR sequences. Thus, 3′UTR sequences that retain m⁶A editing sites substantially enhance RLuc expression in cis while the mutated 3′UTRs lacking m⁶A have no significant positive or negative effect (FIGS. 8B and 8C).

As noted above, m⁶A sites are thought to function by recruiting one or more of the YTHDF proteins to the mRNA. Currently, it remains unclear whether these three proteins are functionally distinct, though our data indicate that all three YTHDFs are recruited to each of the m⁶A editing sites identified on the HIV-1 genome (FIGS. 1 and 2) and we also observed this for the cellular m⁶A editing sites listed in FIG. 4C (data not shown). If this hypothesis is correct, then tethering of the effector domain of YTHDF1, 2 and/or 3 should reproduce the enhancing effect of 3′UTR m⁶A editing sites on RLuc expression seen in FIGS. 4A and C. To test this hypothesis, we expressed fusion proteins consisting of the amino-terminal effector domains of YTHDF1, 2 and 3 linked to the bacteriophage MS2 coat protein. All three FLAG-tagged fusion proteins were expressed at levels comparable to the FLAG-tagged parental YTHDF proteins and showed the same cytoplasmic localization (FIG. 8D). When tested in combination with an RLuc indicator plasmid containing MS2 coat protein binding sites inserted into the 3′UTR, we saw a 3-4-fold enhancement in RLuc expression with all three YTHDF-MS2 fusions when compared to a control GFP-MS2 fusion (FIG. 4F), thus arguing that recruitment of YTHDF proteins to m⁶A editing sites mediates the positive effect of 3′UTRs containing m⁶A sites.

### YTHDF Protein Overexpression Enhances HIV-1 Replication

While the experiments presented in FIG. 4 argue that the recruitment of cellular YTHDF proteins to m⁶A residues present in the 3′UTR of mRNAs can substantially enhance mRNA expression and protein production, these data do not address whether addition of m⁶A also enhances HIV-1 replication. Because the HIV-1 m⁶A editing sites listed in FIG. 3 are located in regions of the viral genome that play key roles in viral replication, e.g., the env/rev gene overlap, the NF-κB repeats and TAR, it is technically difficult to mutate these m⁶A sites without affecting other cis-acting RNA elements and any reduction in viral replication would therefore be very difficult to interpret.

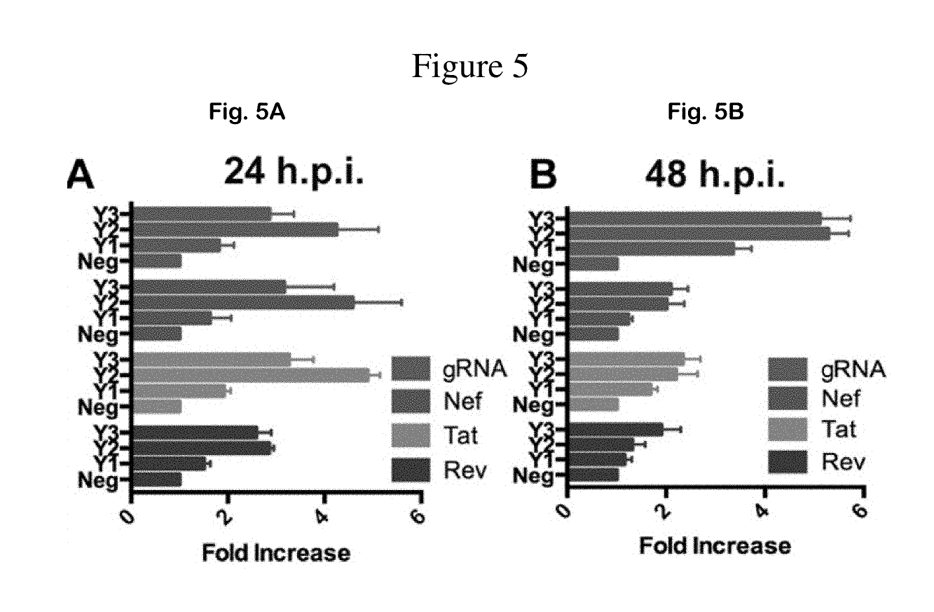

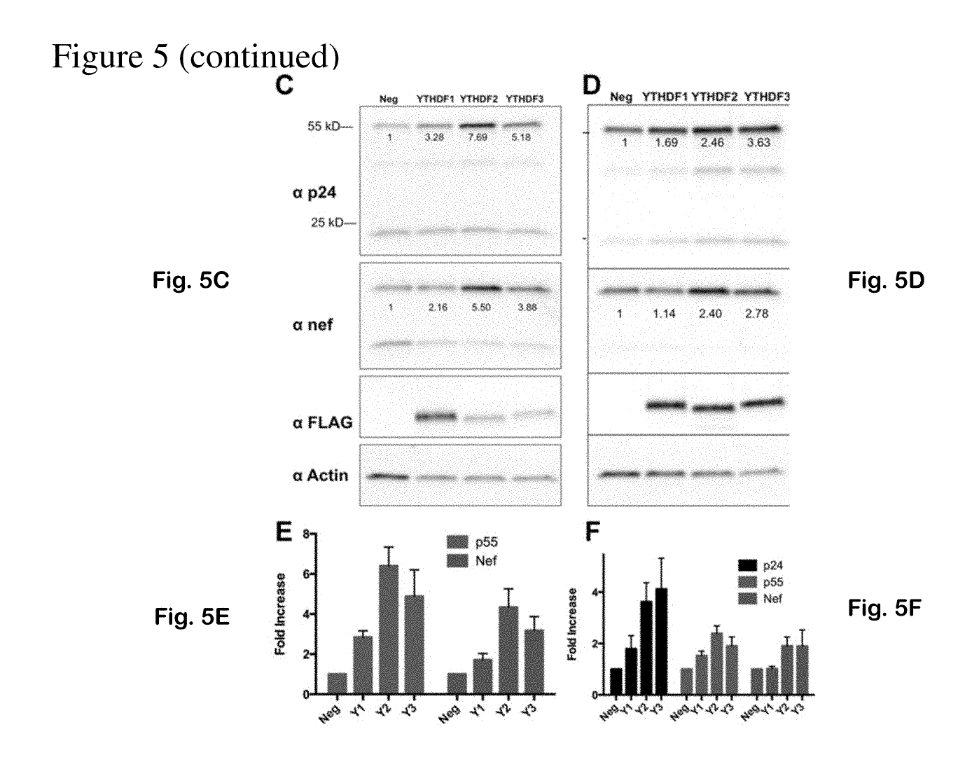

As an alternative approach, we therefore asked whether overexpression of YTHDF1, YTHDF2 or YTHDF3 might enhance HIV-1 gene expression, presumably by facilitating the recruitment of these proteins to viral m⁶A editing sites. As shown in FIGS. 5A and B, overexpression of the YTHDF proteins substantially enhanced the expression of the HIV-1 Nef, Tat and Rev mRNAs as well as the full-length viral genomic RNA (gRNA) at both 24 h and 48 h post-infection (hpi) in human 293T cells. This effect was especially marked when analyzing gRNA expression at 48 hpi, with an ˜6-fold enhancement seen with both YTHDF2 and YTHDF3 overexpression. Analysis of viral protein expression at these same time points (FIGS. 5C, D, E and F) revealed a very similar effect. For example, we observed an ˜6-fold positive effect of YTHDF2 overexpression on p55 Gag expression at 24 hpi and an ˜5-fold effect of YTHDF3 overexpression (FIG. 5E). Similarly, we observed an ˜4-fold increase in HIV-1 p24 capsid expression at 48 hpi for both YTHDF2 and YTHDF3 overexpression. YTHDF1 overexpression exerted a more minor positive effect on viral gene expression (FIGS. 5E and F), though the level of ectopic expression of these three proteins was comparable (FIG. 8D).

We next tested whether overexpression or reduced expression of the YTHDF proteins would affect HIV-1 replication in CD4+ T cells. Western analysis of the expression of these three proteins showed a readily detectable level of expression of YTHDF2, low expression of YTHDF1 and no detectable expression of YTHDF3 in the CD4+ T-cell line CEM-SS (data not shown) and we therefore focused our attention on YTHDF2.

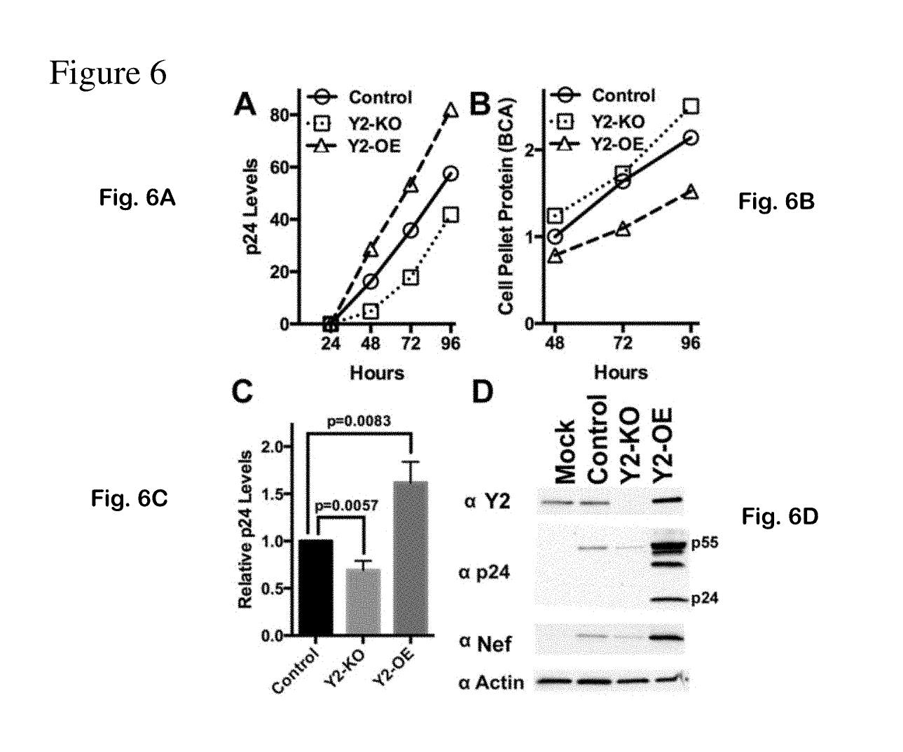

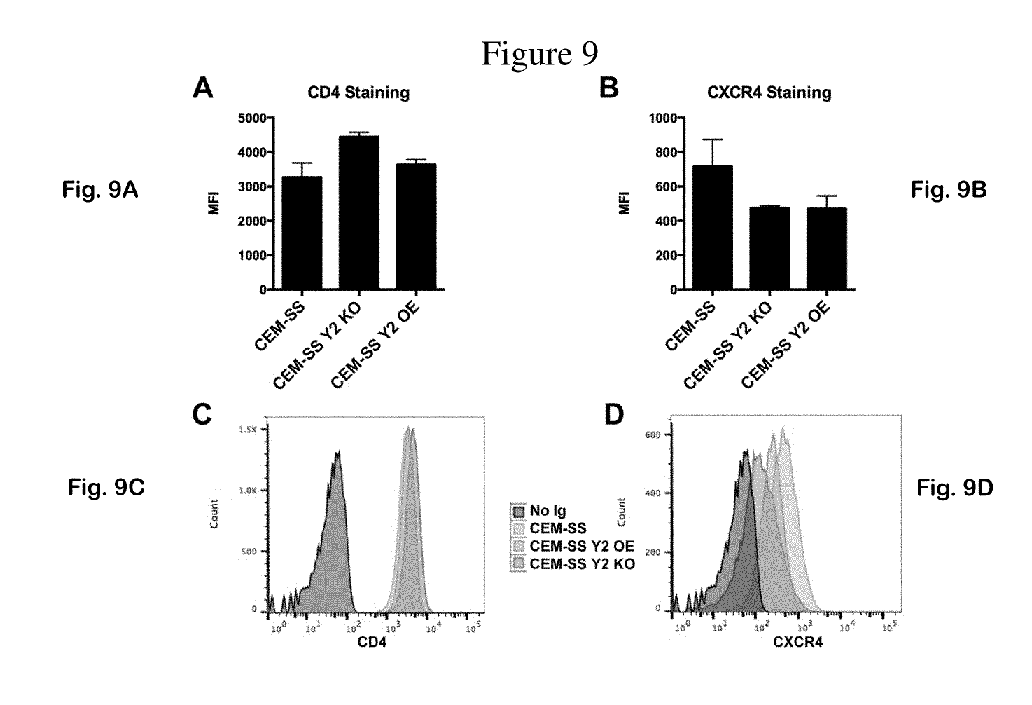

To examine how YTHDF2 affects HIV-1 replication in culture, we generated two subclones of CEM-SS, one in which the endogenous YTHDF2 gene was mutationally inactivated using CRISPR/Cas (Shalem et al., 2014) (Y2-KO) and a second cell line that overexpresses YTHDF2 by ˜2-fold after transduction with a lentiviral YTHDF2 expression vector (Y2-OE). Analysis of these two cell lines, and a control CEM-SS cell line transduced with a GFP-expressing lentivirus, revealed comparable levels of CD4 and CXCR4 expression on their cell surface (FIG. 9). Nevertheless, analysis revealed a significant decline (p<0.006) in viral replication in the Y2-KO CEM-SS cells lacking YTHDF2, and a significant enhancement (p<0.009) in the replication of HIV-1 in the CEM-SS Y2-OE subclone that overexpresses YTHDF2 (FIGS. 6A and C). Of note, this increase in viral replication occurred despite the observation of an enhanced viral cytopathic effect in the Y2-OE culture, which reduced the number of T-cells when compared to the Y2-KO culture (FIG. 6B). Western analysis of viral protein expression at 72 hpi with HIV-1 confirmed a substantially higher level of HIV-1 Gag and Nef expression in the Y2-OE subclone, and a substantially lower level of HIV-1 Gag and Nef expression in the Y2-KO subclone, when compared to the control CEM-SS cells (FIG. 6D). These data therefore further confirm the findings presented in FIG. 4 arguing that the m⁶A-mediated recruitment of YTHDF proteins to viral mRNAs enhances their expression and function and demonstrate that YTHDF protein expression is limiting in both 293T and CEM-SS cells. More importantly, these data argue that m⁶A editing, by recruiting YTHDF proteins to HIV-1 transcripts, significantly enhances the replication potential of HIV-1.

### DISCUSSION

Although m⁶A editing of viral mRNAs was first reported 40 years ago (Krug et al., 1976), the ability to precisely map these editing sites has only recently been achieved. Here, we have focused on the pathogenic human lentivirus HIV-1. We first used an in vitro technique, PA-m⁶A-seq (Chen et al., 2015), to map m⁶A editing sites on the genome of the HIV-1 laboratory strain NL4-3 using an m⁶A-specific antibody (FIG. 1). We then extended these data by using PAR-CLIP (Hafner et al., 2010) to map binding sites for the three human YTHDF reader proteins on the HIV-1 genome in infected cells (FIG. 1). These experiments identified four clusters on the HIV-1 RNA genome that not only bound all three cellular YTHDF proteins independently but also bound the m⁶A-specific antibody (FIG. 1). Analysis of m⁶A editing sites on two primary HIV-1 isolates, BaL and JR-CSF, demonstrated conservation of all four m⁶A binding clusters seen on NL4-3, though interestingly we also detected one (BaL) or two (JR-CSF) novel m⁶A editing sites on these primary isolates (FIG. 2).

Because all the m⁶A editing sites identified on the HIV-1 genome were located proximal to the viral polyadenylation site, in the 3′UTR region of many or all viral mRNAs, we next asked whether substitution of wildtype or m⁶A-deficient forms of the HIV-1 3′ UTR downstream of an indicator gene would affect its expression. As shown in FIGS. 4A to D, we in fact observed a strong positive effect of the HIV-1 3′UTR on indicator gene expression that was entirely lost when the viral m⁶A editing sites were mutated to G. This effect, which was observed in both lymphoid and non-lymphoid cells, was equivalent at both the protein and RNA level, thus suggesting that m⁶A sites stabilize edited mRNAs. Moreover, this effect was not specific for HIV-1 as m⁶A sites derived from human mRNAs exerted a similar positive effect (FIG. 4E). Importantly, we were able to phenocopy the observed enhancement in mRNA function induced by 3′UTR m⁶A sites by recruiting any one of the three human YTHDF proteins to the 3′UTR of an indicator gene by fusion to an RNA binding site derived from the MS2 bacteriophage coat protein (FIG. 4F), thus arguing that m⁶A sites exert their effect by recruiting YTHDF proteins.

Several of the m⁶A editing sites mapped to the HIV-1 RNA genome were localized to sequences that are required for HIV-1 replication for other, unrelated reasons, including the overlap between the env gene and the second coding exon of rev, the LTR NF-κB binding sites and TAR, and mutational perturbation of any one of these would therefore be likely to reduce viral replication, thus making interpretation of any loss of viral fitness upon mutation of the viral m⁶A sites difficult. To circumvent this problem, and given our evidence that m⁶A sites primarily act to recruit YTHDF proteins (FIGS. 1 and 4), we therefore instead asked if overexpression or knockdown of YTHDF proteins, especially YTHDF2, would induce the predicted up or down regulation of HIV-1 replication and gene expression, respectively. As shown most clearly in the CD4+ human T-cell line CEM-SS, we indeed observed a striking increase in HIV-1 replication when YTHDF2 was overexpressed (Y2-OE, FIG. 6) and a marked decline in HIV-1 replication in T cells in which the YTHDF2 gene had been inactivated by DNA editing (Y2-KO, FIG. 6). Together, these data therefore map several m⁶A editing sites to the 3′ UTR region of the HIV-1 genome (FIGS. 1 and 2), reveal that 3′UTR m⁶A editing sites of either viral or cellular origin enhance gene expression in cis (FIG. 4) and finally demonstrate that the level of expression of cellular YTHDF proteins, especially YTHDF2, impacts the level of HIV-1 gene expression and replication in both non-lymphoid (FIG. 5) and lymphoid (FIG. 6) cells in culture, as indeed predicted if the role of m⁶A editing sites is to recruit YTHDF proteins to the mRNA (FIG. 4F).

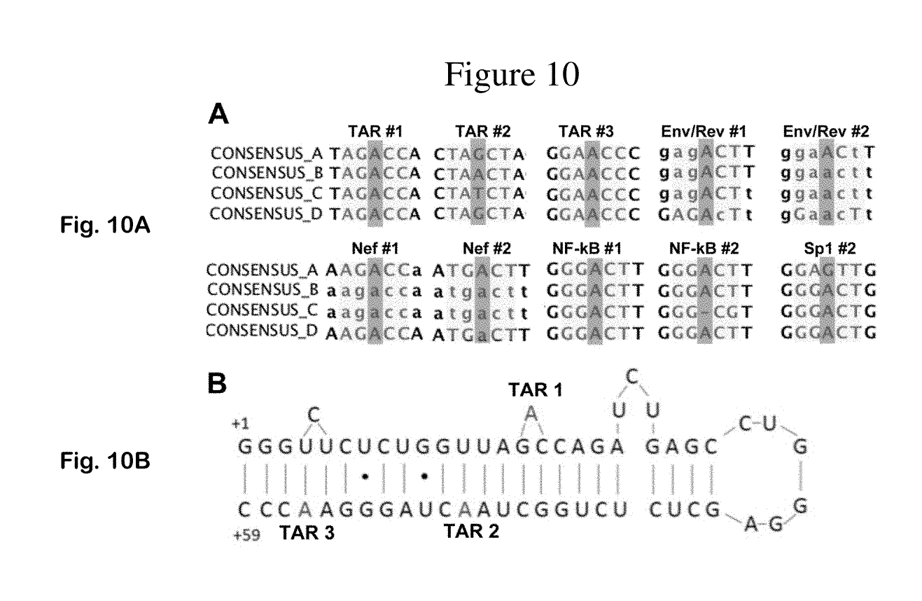

If the m⁶A editing sites in the HIV-1 genome are important for maximizing virus replication, then one would predict that these would be conserved. The four m⁶A editing sites identified in the NL4-3 laboratory strain of HIV-1 were indeed found to be conserved in the primary isolates BaL and JR-CSF, though interestingly these also contained one or two additional m⁶A editing sites not seen in NL4-3 (FIG. 2). As regards the four m⁶A binding clusters mapped in all three HIV-1 variants, these each contain either two or three sites that bear the minimal m⁶A editing consensus 5′-RA*C-3′ (FIG. 3). Analysis of the conservation of these 10 possible m⁶A addition sites across the A, B, C and D clades of HIV-1 (FIG. 10A) shows that 7 out of 10 sites are highly conserved. Two of the three sites in the LTR U3 region are partly conserved, being found in three out of four of these HIV-1 clades, while one site, the second site in TAR, is only weakly conserved. As this potential TAR m⁶A site is a weak consensus editing site, we actually believe that editing at this site is unlikely. Indeed, the large majority of the crosslinking of all three YTHDF proteins to TAR occurs to the 5′ arm of TAR (FIG. 7E), arguing that the optimal m⁶A editing site flanking the bulged A residue at position 17 in the 5′ arm of TAR is the main target for m⁶A editing (FIG. 10B).

The TAR RNA hairpin forms part of the HIV-1 “R” region and is therefore present at both ends of the viral RNA genome (Hauber and Cullen, 1988). Many of the reads obtained during the YTHDF protein PAR-CLIP experiments extend past the R region into U3, thus demonstrating that the 3′ TAR is m⁶A edited (FIG. 7E). While no reads extending past R into U5 were recovered, it remains possible that the TAR m⁶A editing site(s) (FIG. 10) are also utilized at the 5′ end of the HIV-1 genome. This would be of interest given recent data arguing that m⁶A editing sites present in the 5′UTR can induce cap-independent translation, including under conditions of cell stress (Meyer et al., 2015). It has been proposed that HIV-1 mRNAs can also undergo cap independent translation initiation, despite the existence of a cap at the 5′ end of all viral mRNAs, yet no HIV-1 internal ribosome entry site has been identified (Monette et al., 2013). m⁶A editing of the 5′ TAR element might explain this apparent contradiction.

Our observation that m⁶A editing in 3′ UTRs, and the direct recruitment of the YTHDF proteins to 3′UTRs, can significantly enhance the level of mRNA expression and, hence, protein production contrasts with a previous paper arguing that YTHDF2 can destabilize bound mRNAs (Wang et al., 2015). We note, however, that earlier work had suggested that loss of m⁶A correlates with the reduced expression of edited transcripts (Dominissini et al., 2012), which is consistent with our data. While the location of m⁶A residues on a given mRNA, or perhaps their sequence context, could certainly regulate how m⁶A affects mRNA function, we do not believe that the positive effect of m⁶A residues present in the 3′UTR is a unique attribute of HIV-1, as several cellular m⁶A editing sites exerted a similar positive effect (FIG. 4E), as did RNA tethering of any of the three human YTHDF proteins (FIG. 4F). Moreover, it is well established that not only HIV-1, as demonstrated here, but also a wide range of other viruses contain multiple m⁶A editing sites (Dimock and Stoltzfus, 1977; Kane and Beemon, 1985; Krug et al., 1976; Lavi and Shatkin, 1975; Sommer et al., 1976). As viruses are under strong selective pressure to maximize their replication potential, and given that the random mutational inactivation of consensus m⁶A editing site would likely occur at high frequency, the observed conservation of the m⁶A editing sites in HIV-1 argues strongly for a positive role in the viral replication cycle.

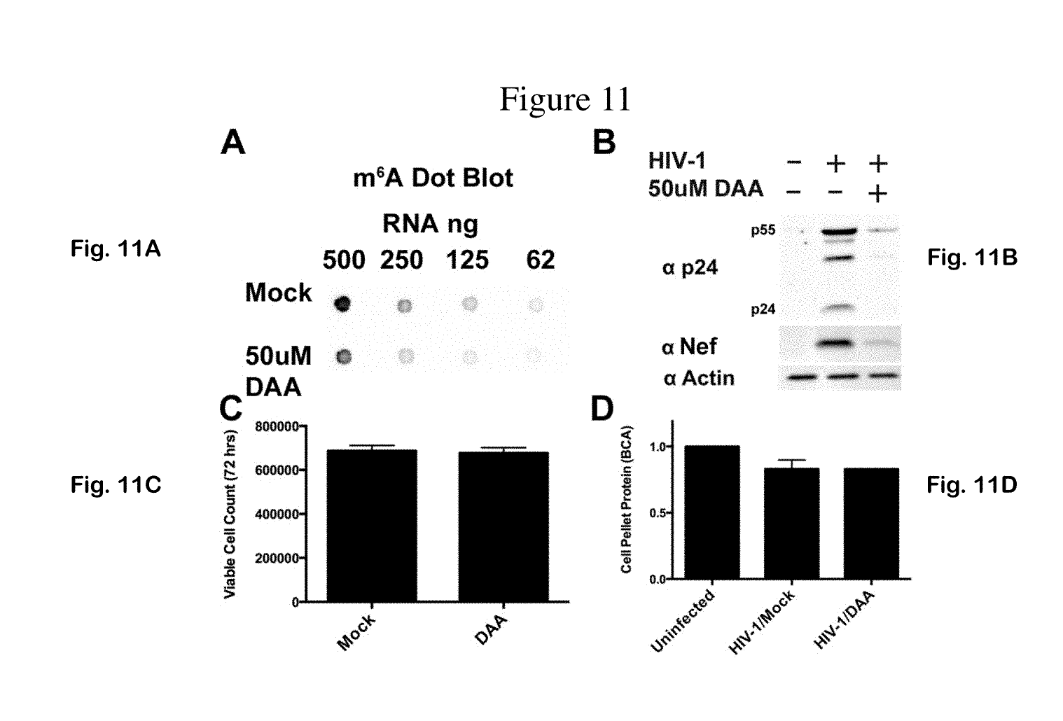

If m⁶A is indeed important for viral replication, then the question arises whether a drug that inhibits m⁶A editing in HIV-1, or indeed other viruses, could act as an effective antiviral. Such a drug does in fact exist. Specifically, 3-deazaadenosine (DAA) has been shown to block m⁶A addition to mRNA substrates by blocking the hydrolysis of S-adenosylhomocysteine, a competitive inhibitor of S-adenosylmethionine, the methyl donor used by the METTL3/METTL14/WTAP complex (Chiang, 1998). Interestingly, DAA has also been reported to inhibit the replication of a range of viruses, including RSV, IAV and HIV-1, all of which display extensive m⁶A editing, though the mechanism of inhibition by DAA has remained uncertain (Bader et al., 1978; Fischer et al., 1990; Flexner et al., 1992). As shown in FIG. 10, we were also able to demonstrate the potent inhibition of HIV-1 replication by DAA. We observed a significant decline in m⁶A levels in total poly(A)+ RNA in cells treated with 50 μM DAA (FIG. 11A), a level that did not reduce cell growth or viability over the four day treatment period (FIGS. 11C and 11D). Remarkably, this same 50 μM level of DAA effectively inhibited the replication of HIV-1 in CEM-SS cells (FIG. 11B), a result which copies the reduction in HIV-1 replication seen in CEM-SS cells lacking a functional YTHDF2 gene (FIG. 6). While these data do not prove that the sole inhibitory mechanism used by DAA to prevent HIV-1 replication in culture revolves around inhibition of m⁶A editing, they are intriguing in that they suggest that drugs that reduce m⁶A editing might have the potential to inhibit the replication of not only HIV-1 but also other viral pathogens, such as IAV.

### Experimental Procedures

Western blots used the following primary antibodies: HIV-1 p24 (AIDS Reagent Program-3517), YTHDF2 (SC-162427, Santa Cruz), Actin (SC-4/778, Santa Cruz), FLAG (F1804, Sigma) and HIV-1 Nef (AIDS Reagent Program-2949). ELISAs utilized an HIV-1 p24 antigen capture kit (ABL Catalog #5421 and 5447). Total poly(A)+ RNA was purified using Ambion Poly(A)Purist MAG kits.

### Molecular Clones

cDNAs encoding full length, FLAG-tagged forms of the three YTHDF proteins were obtained by PCR from a human cDNA library and were then used to generate pLEX-based lentiviral vectors. For the YTHDF-MS2 coat protein fusions, pcDNA3 was modified to express pcGFP/MS2, pcYTHDF1/MS2, pcYTHDF2/MS2 and pcYTHDF3/MS2 chimeric proteins using the same YTHDF templates. The coordinates of the included N-terminal YTHDF segments are as follows: YTHDF1 (1-382), YTHDF2 (1-401), and YTHDF3 (1-409). The open reading frame for the MS2 bacteriophage coat protein was PCR amplified from pMS2-p65-HSF, (Addgene, #61426). Four copies of the MS2 RNA aptamer were inserted into psiCHECK2 (Promega) to generate the psiCHECK2-4XMS2 reporter plasmid. For the m⁶A site indicator plasmids, inserts were synthesized with predicted methyl receptor adenosines mutated to a guanosine. These m⁶A site mutant inserts, and the analogous WT inserts, were then cloned into psiCHECK2 (Promega) via the XhoI and NotI sites. The HIV-1 U3/NF-κB/TAR insert starts 34 bp 5′ of NF-κB site II in U3 and spans the entire R region, including TAR, before terminating 26 bp into the LTR U5 region. The 3′UTR construct has an identical 3′ terminus and initiates at the BamHI site in pNL4-3. All cellular m⁶A indicator constructs were constructed by insertion of oligonucleotides encompassing full length cellular m⁶A acceptor sites, in their wildtype or mutated form, into the 3′UTR of RLuc in psiCheck2.

### Cell Culture, HIV-1 Production, and Infections

293T cells were cultured in Dulbecco's Modified Eagle Medium (DMEM) containing 10% fetal bovine serum (FBS) and antibiotics. CEM-SS cells were cultured in RPMI 1640 containing 10% FBS and antibiotics. HIV-1 was produced by transfection of 293T cells with the pNL4-3 molecular clone; at 72 h post-transfection, supernatant media were harvested, clarified by centrifugation and then filtered through a 0.45 μM filter (PALL). To prepare vesicular stomatitis virus glycoprotein (VSV-G) pseudotyped virus, pVSV-G was transfected at a 1:10 ratio relative to an HIV-1 proviral expression vector encoding NL4-3, BaL or JR-CSF. The supernatant media were harvested 72 h later, as described above. 293T cells were infected with the HIV-1 virus stock overnight and fresh media added the next morning. CEM-SS sub-clones were HIV-1 infected overnight, then washed with PBS and resuspended in fresh media next morning. Samples for p24 ELISA and Western analysis were collected over time from 6 ml infections per condition/biological replicate.

### 293T and CEM-SS Clonal Cell Lines

Clonal YTHDF expressing 293T cell lines were produced by transduction with a constitutive lentiviral YTHDF expression vector followed by selection for the encoded puromycin resistance marker. Resistant cells were then sub-cloned by limiting dilution. CEM-SS (NIH AIDS Reagent Program catalog #776) overexpressing YTHDF2 were also obtained by lentiviral transduction, and puromycin resistant cells then sub-cloned by limiting dilution. YTHDF2 overexpression was confirmed by Western. YTHDF2 knockout CEM-SS cells were obtained by transduction with lentiCRISPRv2, with the sgRNA sequence 5′-GGAACCTTACTTGAGTCCAC-3′, obtained from a published library (Shalem et al., 2014), and were cloned by limiting dilution. The control for these cell lines was a puromycin selected GFP-expressing CEM-SS sub-clone.

### PAR-CLIP and PA-m⁶A-Seq

PAR-CLIP was performed as described (Hafner et al., 2010). The three clonal 293T cell lines expressing FLAG-YTHDF proteins, or FLAG-GFP as a control, were infected with HIV-1 NL4-3 pseudotyped with VSV-G, incubated for 48 h and then pulsed with 100 μM 4SU in fresh media for 16 h. The cells were then harvested and the PAR-CLIP protocol performed. JR-CSF and BaL infections were conducted similarly. CEM-SS cells were infected with HIV-1, 4SU pulsed, total poly(A)+ RNA purified, and the rest of the PA-m⁶A-Seq protocol performed as described using an m⁶A specific polyclonal antibody (SySy). For the indicator plasmid PAR-CLIP experiment shown in FIG. 8A, 293T cells expressing FLAG-YTHDF2 were transfected with psiCHECK2-based constructs containing wildtype or mutant forms of the U3/NF-kB/TAR sequence. After 48 h, the cells were pulsed for 16 h with 4SU and harvested for PAR-CLIP.

PAR-CLIP libraries were sequenced on a HiSeq 2000, base calling was performed with CASAVA and processed with the fastx toolkit (available at hannonlab.cshl.edu/fastx_toolkit). Reads >14 bp in length were used for bioinformatic analysis. All alignments were performed with Bowtie (Langmead et al., 2009). Reads were initially aligned to the human genome build hg19 allowing up to 1 mismatch, and unaligned reads were then aligned to the HIV-1 genome of interest, again with 1 mismatch. The HIV-1 aligned reads exhibited a substantial enrichment of reads containing T>C mutations when derived from cells expressing one of the YTHDF proteins (FIG. 7C), and these reads were of mean length >24 nt (FIG. 7D). For all visualizations, only reads containing T>C mutations were considered. Data was processed with in-house Perl scripts and Samtools, and visualized with IGV (Li et al., 2009; Robinson et al., 2011).

The raw sequencing data obtained from small RNA deep sequencing have been submitted to the NCBI Gene Expression Omnibus (GEO) and are available through accession number GSE77890.

### Indicator Assays and MS2-Tethering

HIV-1 based indicators were transfected into 293T or CEM-SS cells utilizing the polyethylenimine (PEI) and Lipofectamine LTX (Invitrogen) transfection methods, respectively. Cells were harvested 48 h later and subjected to either cell lysis using Passive Lysis Buffer-PLB (Promega Dual Luciferase Kit), for protein extraction, or using TRIzol, for total RNA extraction. Protein lysates were analyzed for RLuc and FLuc levels using a Dual Luciferase Assay Kit (Promega). Total RNA was reverse-transcribed using a SuperScript III kit (Invitrogen) followed by SYBR green qPCR of cDNAs utilizing RLuc, FLuc, and GAPDH mRNA specific primers. RLuc mRNA abundance was determined by normalizing first to the endogenous GAPDH mRNA and then to the control FLuc mRNA. For the tethering assays, 293T cells were transfected with 50 ng psiCHECK2 or the psiCHECK2-4xMS2 reporter and 500 ng pcGFP/MS2, pcYTHDF1/MS2, pcYTHDF2/MS2 or pcYTHDF3/MS2 using PEI. Cells were harvested 72 h post-transfection and analyzed for RLuc (reporter) and FLuc (internal control) activity using the Dual-Luciferase Assay.

### Example 2

### YTHDF2 Over-Expression Enhances IAV and AAV Growth

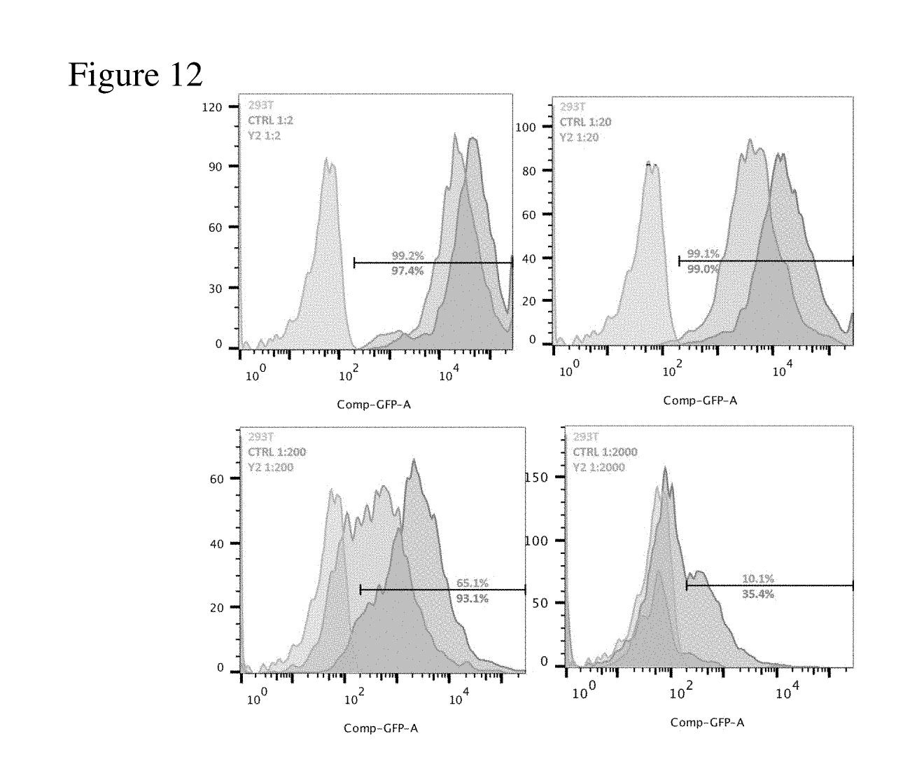

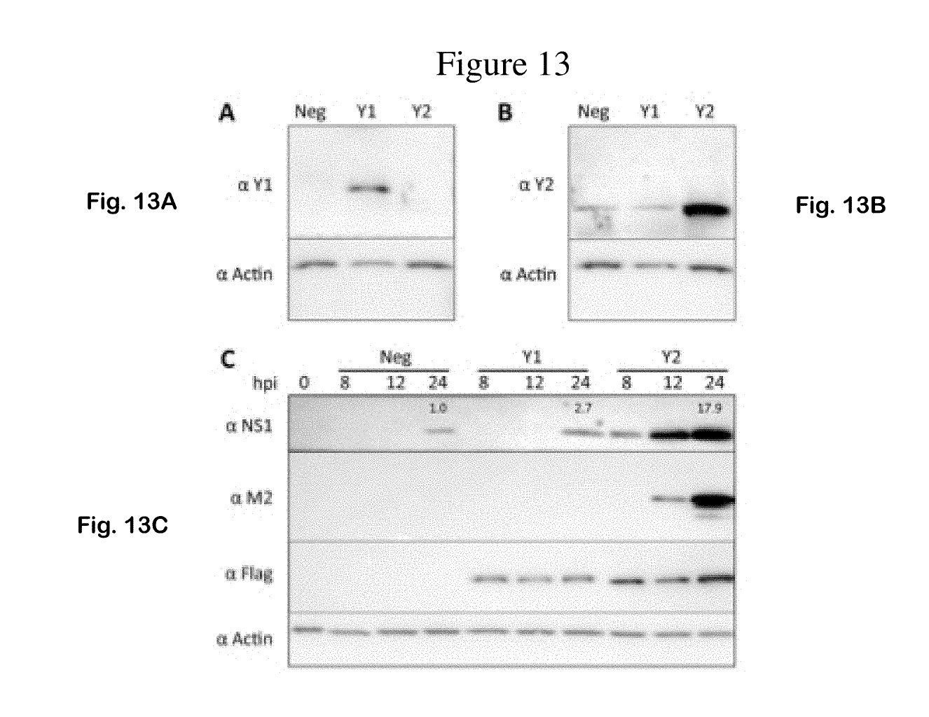

While studying the role of methylation of adenosine at the N6 position (m⁶A), we noted that over-expression of the human protein YTHDF2 in the human cell line 293T, and to a lesser extent overexpression of the related human proteins YTHDF1 and YTHDF3, substantially enhanced the production of HIV-1 viral proteins and mRNAs in this cell line (See, e.g., FIG. 5). We were therefore curious as to whether this implied that YTHDF2 over-expression might also promote the expression of viral proteins encoded by other viral species. We have now shown that Adeno Associated Virus (AAV) vector production is enhanced by 5-fold or more in 293T cells expressing YTHDF2 compared to wild type cells (Table 1) and similar data were obtained using an AAV vector encoding green fluorescent protein (GFP) (FIG. 12). Moreover, we have now generated clones of the human cell line A549 over-expressing YTHDF2 and have shown that influenza A virus expresses far higher levels of viral proteins in the YTHDF2 over-expessing cell line (FIG. 13). Therefore, cells over-expressing YTHDF2, or possibly YTHDF3, could be used to produce higher titers of lentiviral vectors based on HIV-1 or other lentiviruses as well as higher titers of AAV, which is routinely produced in 293T cells. This could be done using clonal cells stably over-expressing YTHDF2, as here, or by co-transfection of a YTHDF2 expression plasmid. Similarly, A549 cells, or other cell lines like Vero, engineered to overexpress YTHDF2 could be used to grow vaccine strains of influenza A virus (IAV) at much higher titers than currently achievable. As YTHDF2 increases the production of a retrovirus (HIV-1), a single-stranded DNA virus (AAV) and a single stranded RNA virus (IAV) we believe it is likely that a wide range of viruses, and vaccine strains derived from them, would replicate better in cells over-expressing YTHDF2. Therefore, this could represent a very useful method to increase commercial production of viral vectors and vaccines.

TABLE 1
Luciferase from AAV-infected T cells 72 hours post-infection.
An AAV vector encoding luciferase was packaged in wildtype 293T
cells (CTRL) or in 293T cells overexpressing YTHDF2. Virus was
harvested, purified using a sucrose gradient and then used to infect naïve,
wildtype 293T cells. These were lysed 48 h later and luciferase levels
determined. Note that the level of luciferase is consistently ~5 fold
higher when the packaging cells expressed YTHDF2.

| Packaging Cells | Luciferase Expression | Fold Increase |
|---|---|---|
| CTRL (1:2) | 5,860,650 | |
| YTHDF2 (1:2) | 26,610,600 | 4.5x |
| CTRL (1:20) | 1,690,831 | |
| YTHDF2 (1:20) | 6,727,420 | 4.0x |
| CTRL (1:200) | 262,411 | |
| YTHDF2 (1:200) | 1,186,033 | 4.5x |
| CTRL (1:2,000) | 20,258 | |
| YTHDF2 (1:2,000) | 98,886 | 4.9x |
| CTRL (1:20,000) | 2,024 | |
| YTHDF2 (1:20,000) | 8,303 | 4.1x |
| CTRL (1:200,000) | 189 | |
| YTHDF2 (1:200,000) | 972 | 5.1x |

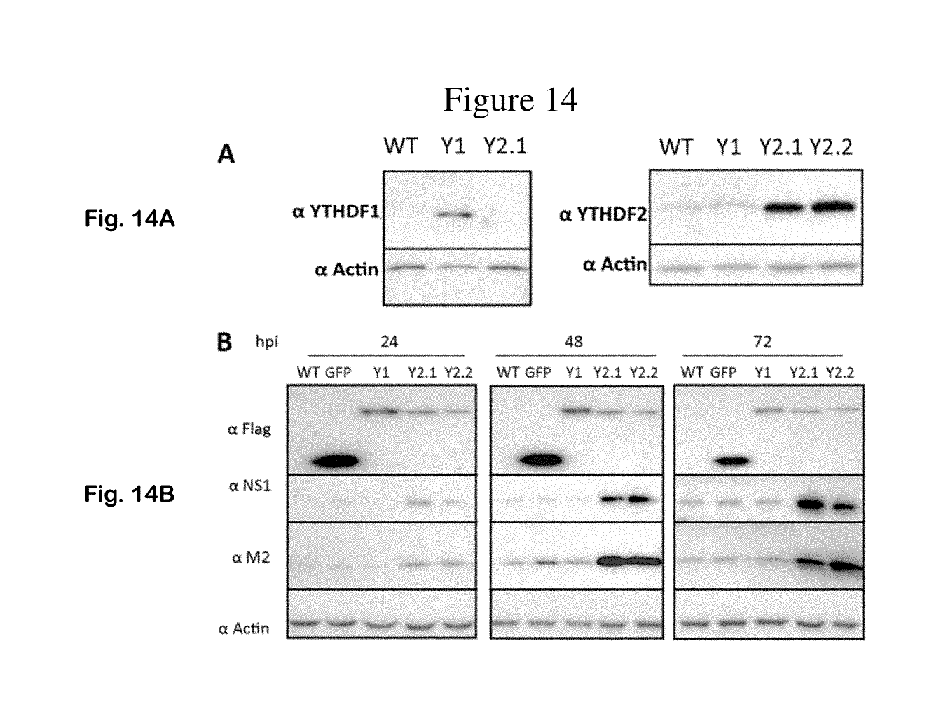

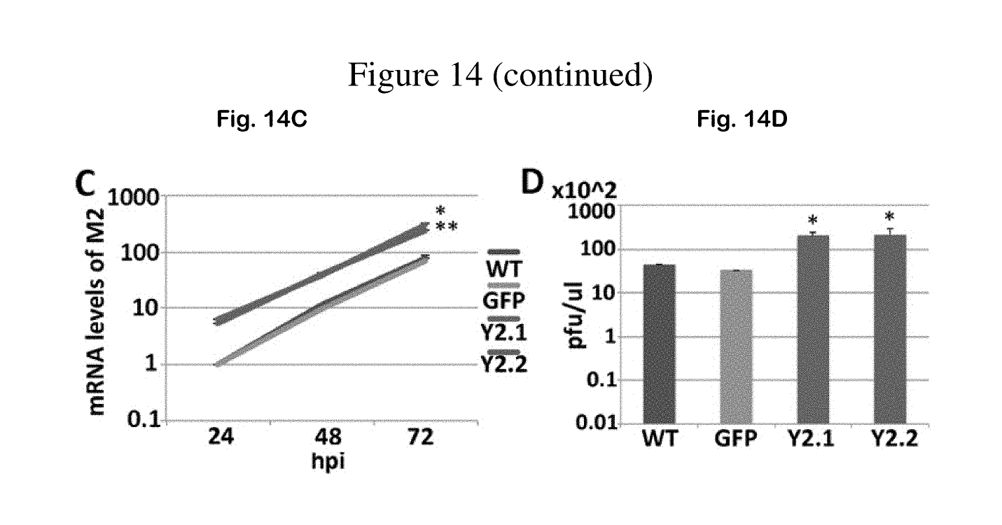

To address the unlikely possibility that the greatly increased expression of IAV proteins seen in A549 cells expressing ectopic human YTHDF1 (FIG. 13) was some form of clonal artifact, we generated a second, independent clone of A549 also expressing ectopic FLAG-tagged YTHDF2 which we named Y2.2 (the original clone was renamed Y2.1). As determined by Western blot using an antiserum specific for YTHDF2 (FIG. 14A, right panel) the Y2.2 cells expressed a level of YTHDF2 that was much higher than seen in control WT cells and comparable to what was detected in the original Y2.1 cell line. We next analyzed the ability of IAV strain PR8 to replicate in the parental, WT cells, in A549 cells engineered to express an irrelevant control protein (FLAG-tagged green fluorescent protein GFP), YTHDF1 or YTHDF2. Unlike the experiment shown in FIG. 13, which used a high MOI of 1.0 and was performed in the absence of trypsin, thus blocking IAV spread, this new experiment used a very low MOI of 0.01 of IAV strain PR8 and the A549 cells were cultured in the presence of trypsin, which cleaves the IAV HA glycoprotein and thereby allows IAV spread to occur. Infected cultures were harvested at 24, 48 and 72 hours post-infection (hpi) and used for Western blot analysis (FIG. 14B) and determination of viral mRNA levels by qRT-PCR (FIG. 14C). As can be readily observed, we again detected a substantially higher level of expression of the viral NS1 and M2 proteins at all time points after infection in the YTHDF2-expressing A549 cell clones (FIG. 14B) and we also detected a similar increase in the level of M2 mRNA expression (FIG. 14C), which proved to be statistically significant. At 72 hpi, we also harvested one replicate of each infection condition and directly measured the level of IAV infectious particle production by plaque assay on MDCK cells (FIG. 14D). As expected, this also revealed an almost 10-fold increase in infectious IAV in the two A549 cultures expressing ectopic YTHDF2, as compared to the two control cultures.

### REFERENCES

Adachi, A., Gendelman, H. E., Koenig, S., Folks, T., Willey, R., Rabson, A., and Martin, M. A. (1986). Production of acquired immunodeficiency syndrome-associated retrovirus in human and nonhuman cells transfected with an infectious molecular clone. J Virol 59, 284-291.
Bader, J. P., Brown, N. R., Chiang, P. K., and Cantoni, G. L. (1978). 3-Deazaadenosine, an inhibitor of adenosylhomocysteine hydrolase, inhibits reproduction of Rous sarcoma virus and transformation of chick embryo cells. Virology 89, 494-505.
Cann, A. J., Zack, J. A., Go, A. S., Arrigo, S. J., Koyanagi, Y., Green, P. L., Koyanagi, Y., Pang, S., and Chen, I. S. (1990). Human immunodeficiency virus type 1 T-cell tropism is determined by events prior to provirus formation. J Virol 64, 4735-4742.
Carlile, T. M., Rojas-Duran, M. F., Zinshteyn, B., Shin, H., Bartoli, K. M., and Gilbert, W. V. (2014). Pseudouridine profiling reveals regulated mRNA pseudouridylation in yeast and human cells. Nature 515, 143-146.
Chen, K., Lu, Z., Wang, X., Fu, Y., Luo, G. Z., Liu, N., Han, D., Dominissini, D., Dai, Q., Pan, T., et al. (2015). High-resolution N(6)-methyladenosine (m(6) A) map using photo-crosslinking-assisted m(6) A sequencing. Angew Chem Int Ed Engl 54, 1587-1590.
Chiang, P. K. (1998). Biological effects of inhibitors of S-adenosylhomocysteine hydrolase. Pharmacol Ther 77, 115-134.
Desrosiers, R. C., Friderici, K. H., and Rottman, F. M. (1975). Characterization of Novikoff hepatoma mRNA methylation and heterogeneity in the methylated 5′ terminus. Biochemistry 14, 4367-4374.
Dimock, K., and Stoltzfus, C. M. (1977). Sequence specificity of internal methylation in B77 avian sarcoma virus RNA subunits. Biochemistry 16, 471-478.
Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m⁶A RNA methylomes revealed by m⁶A-seq. Nature 485, 201-206.
Dominissini, D., Nachtergaele, S., Moshitch-Moshkovitz, S., Peer, E., Kol, N., Ben-Haim, M. S., Dai, Q., Di Segni, A., Salmon-Divon, M., Clark, W. C., et al. (2016). The dynamic N-methyladenosine methylome in eukaryotic messenger RNA. Nature 530, 441-446.
Fischer, A. A., Muller, K., and Scholtissek, C. (1990). Specific inhibition of the synthesis of influenza virus late proteins and stimulation of early, M2, and NS2 protein synthesis by 3-deazaadenosine. Virology 177, 523-531.
Flexner, C. W., Hildreth, J. E., Kuncl, R. W., and Drachman, D. B. (1992). 3-Deaza-adenosine and inhibition of HIV. Lancet 339, 438.
Hafner, M., Landthaler, M., Burger, L., Khorshid, M., Hausser, J., Berninger, P., Rothballer, A., Ascano, M., Jr., Jungkamp, A. C., Munschauer, M., et al. (2010). Transcriptome-wide identification of RNA-binding protein and microRNA target sites by PAR-CLIP. Cell 141, 129-141.
Hauber, J., and Cullen, B. R. (1988). Mutational analysis of the trans-activation-responsive region of the human immunodeficiency virus type I long terminal repeat. J Virol 62, 673-679.
Hwang, S. S., Boyle, T. J., Lyerly, H. K., and Cullen, B. R. (1991). Identification of the envelope V3 loop as the primary determinant of cell tropism in HIV-1. Science 253, 71-74.
Jia, G., Fu, Y., Zhao, X., Dai, Q., Zheng, G., Yang, Y., Yi, C., Lindahl, T., Pan, T., Yang, Y. G., et al. (2011). N6-methyladenosine in nuclear RNA is a major substrate of the obesity-associated FTO. Nat Chem Biol 7, 885-887.
Kane, S. E., and Beemon, K. (1985). Precise localization of m⁶A in Rous sarcoma virus RNA reveals clustering of methylation sites: implications for RNA processing. Mol Cell Biol 5, 2298-2306.
Ke, S., Alemu, E. A., Mertens, C., Gantman, E. C., Fak, J. J., Mele, A., Haripal, B., Zucker-Scharff, I., Moore, M. J., Park, C. Y., et al. (2015). A majority of m⁶A residues are in the last exons, allowing the potential for 3′ UTR regulation. Genes Dev 29, 2037-2053.
Krug, R. M., Morgan, M. A., and Shatkin, A. J. (1976). Influenza viral mRNA contains internal N6-methyladenosine and 5′-terminal 7-methylguanosine in cap structures. J Virol 20, 45-53.
Langmead, B., Trapnell, C., Pop, M., and Salzberg, S. L. (2009). Ultrafast and memory-efficient alignment of short DNA sequences to the human genome. Genome Biol 10, R25.
Lavi, S., and Shatkin, A. J. (1975). Methylated simian virus 40-specific RNA from nuclei and cytoplasm of infected BSC-1 cells. Proc Natl Acad Sci USA 72, 2012-2016.
Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
Lichinchi, G., Gao, S., Saletore, Y., Gonzalez, G. M., Bansal, V., Wang, Y., Mason, C. E., and Rana, T. M. (2016). Dynamics of the human and viral m⁶A RNA methylomes during HIV-1 infection of T cells. Nat Microbiol doi:10.1038/nmicrobiol.2016.11, 16011.
Liu, J., Yue, Y., Han, D., Wang, X., Fu, Y., Zhang, L., Jia, G., Yu, M., Lu, Z., Deng, X., et al. (2014). A METTL3-METTL14 complex mediates mammalian nuclear RNA N6-adenosine methylation. Nat Chem Biol 10, 93-95.
Meyer, K. D., and Jaffrey, S. R. (2014). The dynamic epitranscriptome: N6-methyladenosine and gene expression control. Nat Rev Mol Cell Biol 15, 313-326.
Meyer, K. D., Patil, D. P., Zhou, J., Zinoviev, A., Skabkin, M. A., Elemento, O., Pestova, T. V., Qian, S. B., and Jaffrey, S. R. (2015). 5′ UTR m(6)A Promotes Cap-Independent Translation. Cell 163, 999-1010.
Meyer, K. D., Saletore, Y., Zumbo, P., Elemento, O., Mason, C. E., and Jaffrey, S. R. (2012). Comprehensive analysis of mRNA methylation reveals enrichment in 3′ UTRs and near stop codons. Cell 149, 1635-1646.
Monette, A., Valiente-Echeverria, F., Rivero, M., Cohen, E. A., Lopez-Lastra, M., and Mouland, A. J. (2013). Dual mechanisms of translation initiation of the full-length HIV-1 mRNA contribute to gag synthesis. PLoS One 8, e68108.
Robinson, J. T., Thorvaldsdottir, H., Winckler, W., Guttman, M., Lander, E. S., Getz, G., and Mesirov, J. P. (2011). Integrative genomics viewer. Nat biotechnol 29, 24-26.
Schwartz, S., Bernstein, D. A., Mumbach, M. R., Jovanovic, M., Herbst, R. H., Leon-Ricardo, B. X., Engreitz, J. M., Guttman, M., Satija, R., Lander, E. S., et al. (2014). Transcriptome-wide mapping reveals widespread dynamic-regulated pseudouridylation of ncRNA and mRNA. Cell 159, 148-162.
Shalem, O., Sanjana, N. E., Hartenian, E., Shi, X., Scott, D. A., Mikkelsen, T. S., Heckl, D., Ebert, B. L., Root, D. E., Doench, J. G., et al. (2014). Genome-scale CRISPR-Cas9 knockout screening in human cells. Science 343, 84-87.
Sommer, S., Salditt-Georgieff, M., Bachenheimer, S., Darnell, J. E., Furuichi, Y., Morgan, M., and Shatkin, A. J. (1976). The methylation of adenovirus-specific nuclear and cytoplasmic RNA. Nucleic Acids Res 3, 749-765.
Squires, J. E., Patel, H. R., Nousch, M., Sibbritt, T., Humphreys, D. T., Parker, B. J., Suter, C. M., and Preiss, T. (2012). Widespread occurrence of 5-methylcytosine in human coding and noncoding RNA. Nucleic Acids Res 40, 5023-5033.
Wang, X., Lu, Z., Gomez, A., Hon, G. C., Yue, Y., Han, D., Fu, Y., Parisien, M., Dai, Q., Jia, G., et al. (2014). N6-methyladenosine-dependent regulation of messenger RNA stability. Nature 505, 117-120.
Wang, X., Zhao, B. S., Roundtree, I. A., Lu, Z., Han, D., Ma, H., Weng, X., Chen, K., Shi, H., and He, C. (2015). N(6)-methyladenosine Modulates Messenger RNA Translation Efficiency. Cell 161, 1388-1399.
Yue, Y., Liu, J., and He, C. (2015). RNA N6-methyladenosine methylation in post-transcriptional gene expression regulation. Genes Dev 29, 1343-1355.
Zheng, G., Dahl, J. A., Niu, Y., Fedorcsak, P., Huang, C. M., Li, C. J., Vagbo, C. B., Shi, Y., Wang, W. L., Song, S. H., et al. (2013). ALKBH5 is a mammalian RNA demethylase that impacts RNA metabolism and mouse fertility. Mol Cell 49, 18-29.

1. A construct comprising a promoter operably connected to a polynucleotide encoding a polypeptide, wherein the polynucleotide comprises at least two engineered m⁶A sequences.

2. A construct comprising in the 5′ to 3′ direction of at least one strand of the construct, a heterologous coding sequence encoding a heterologous polypeptide, and a UTR sequence, wherein the UTR sequence comprises at least two m⁶A sequences.

3.-9. (canceled)

10. A construct comprising a promoter operably connected to a polynucleotide comprising an insert site or encoding a heterologous polypeptide, and at least one m⁶A sequence.

11. The construct of claim 10, further comprising a polyA site.

12. The construct of claim 11, wherein the construct comprises, in the 5′ to 3′ direction of at least one strand of the construct, the promoter, the polynucleotide, the at least one m⁶A sequence and the polyA site.

13.-21. (canceled)

22. A vector comprising the construct of claim 10.

23. (canceled)

24. A cell comprising the construct of claim 10.

25. (canceled)

26. (canceled)

27. The cell of claim 24, wherein the cell overexpresses a YTHDF polypeptide.

28. (canceled)

29. A method for producing a heterologous polypeptide in a cell comprising introducing the construct of claim 10 into the cell.

30. (canceled)

31. (canceled)

32. The method of claim 29, further comprising expressing a YTHDF polypeptide in the cell.

33.-40. (canceled)

41. A construct comprising (i) a heterologous coding sequence encoding a heterologous polypeptide, and (ii) a UTR sequence, the UTR sequence comprising at least one RNA-binding polypeptide recognition sequence.

42.-45. (canceled)

46. A cell comprising:
(a) a fusion protein comprising a YTHDF polypeptide and a RNA-binding polypeptide, and
(b) the construct of claim 41.

47. (canceled)

48. (canceled)

49. A method for producing a heterologous polypeptide in a cell comprising:
(a) expressing a fusion protein comprising a YTHDF polypeptide and a RNA-binding polypeptide in the cell,
(b) introducing or expressing the construct of claim 41 in the cell.

50.-54. (canceled)

55. A cell engineered to overexpress a YTHDF polypeptide.

56. The cell of claim 55, further comprising a virus or construct comprising at least one m⁶A sequence.

57. (canceled)

58. The cell of claim 56, wherein the virus is selected from the group consisting of a measles virus, a mumps virus, a rubella virus, an influenza virus, a varicella-zoster virus, a polio virus, a rotavirus, a yellow fever virus, a retrovirus, an adenovirus, a herpes simplex virus and a rabies virus.

59.-63. (canceled)

64. A method of producing a virus in the cell of claim 55 comprising introducing the virus into the cell, wherein the virus comprises at least one m⁶A sequence.

65. The method of claim 64, wherein the YTHDF polypeptide is selected from the group consisting of SEQ ID NO: 4, SEQ ID NO: 5, SEQ ID NO: 6, and a polypeptide having at least 80% sequence identity to SEQ ID NO: 4, SEQ ID NO: 5, or SEQ ID NO: 6.

66. The method of claim 64, wherein the virus is selected from the group consisting of a measles virus, a mumps virus, a rubella virus, an influenza virus, a varicella-zoster virus, a polio virus, a rotavirus, a yellow fever virus, a retrovirus, an adenovirus, a herpes simplex virus and a rabies virus.

67.-69. (canceled)

70. The method of claim 64, wherein the cell is selected from the group consisting of a mammalian cell, a chicken cell, or an insect cell.

71. (canceled)


SEQUENCE LISTING (40 sequences)

SEQ ID NO: 1 - DNA, 1188 bp, Human immunodeficiency virus 1
  misc_feature (1)..(1188): NL4-3
ggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtg

SEQ ID NO: 2 - DNA, 1244 bp, Human immunodeficiency virus 1
  misc_feature (1)..(1244): JRCSF
gagacagatccggacaattagtgaacggattcttagcacttatctgggtcgacctgcggagcctgttcctcttcagctaccaccgcttgagagacttactcttgactgtaacgaggattgtggaacttctgggacgcagggggtgggaaatcctgaaatactggtggaatctcctacagtattggagtcaggaactaaagaatagtgctgttagcttgcttaatgccacagctatagcagtagctgaggggacagataggattatagaagtagtacaaagagtttatagggctattctccacatacctacaagaataagacagggcttggaaagggctttgctataagatgggtggcaagtggtcaaaacatagtgtgcctggatggtctactgtaagggaaagaatgagacgagctgagccagcaacagatagggtgagacaaactgagccagcagcagtaggggtgggagcagtatctcgagacctggaaaaacatggagcaatcacaagtagcaatacagcagctaccaatgctgattgtgcctggctagaagcatatgaggatgaggaagtgggttttccagtcagacctcaggtacctttaagaccaatgacttacaaggcagctatagatcttagccactttttaaaagaaaaggggggactggaagggctaatttactcacagaaaagacaagatatccttgatctgtggatctaccacacacaaggctacttccctgattggcagaactacacagcaggaccaggggtcagatttccactgacctttggatggtgcttcaagctagtaccagttgatccagagaaggtagaagaggccaatgaaggagagaacaactgcttgttacaccctatgagccagcatggaatggacgacccagagaaggaagtgttagtgtggaagtttgacagcaagctagcattgcatcacgtggcccgagagctgcatccggagtactacaaggactgctgacaccgagctttctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctagctagggaacccactgcttaagcctcaataaagcttgccttgagtgcttcaagtagtgtgtgcccgtctgttgtgtg

SEQ ID NO: 3 - DNA, 1188 bp, Human immunodeficiency virus 1
  misc_feature (1)..(1188): BaL
ggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttatagagctattcgccacatacctagaagaataagacagggcttggaaaggattttgctataagatgggtggcaagtggtcaaaaagtagtgtggttggatggcctgctgtaagggaaagaatgagacgagctgagccagcagcagatggggtgggagcagcatctcgagacctagaaaaacatggagcaatcacaagtagcaacacagcagctaacaatgctgcttgtgcctggctagaagcacaagaggaggagaaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccagggatcagatatccactgacctttggatggcgctacaagctagtaccagttgagccagagaagttagaagaagccaacaaaggagagaacaccagcttgttacaccctgtgagcctgcatggaatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtg

SEQ ID NO: 4 - protein, 559 aa, Homo sapiens
  misc_feature (1)..(559): Human YTHDF1
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 5 - protein, 579 aa, Homo sapiens
  misc_feature (1)..(579): Human YTHDF2
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASNVPKVVGSAVGSGSITSNIVASNSLPPATIAPPKPASWADIASKPAKQQPKLKTKNGIAGSSLPPPPIKHNMDIGTWDNKGPVAKAPSQALVQNIGQPTQGSPQPVGQQANNSPPVAQASVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 6 - protein, 585 aa, Homo sapiens
  misc_feature (1)..(585): Human YTHDF3
MSATSVDQRPKGQGNKVSVQNGSIHQKDAVNDDDFEPYLSSQTNQSNSYPPMSDPYMPSYYAPSIGFPYSLGEAAWSTAGDQPMPYLTTYGQMSNGEHHYIPDGVFSQPGALGNTPPFLGQHGFNFFPGNADFSTWGTSGSQGQSTQSSAYSSSYGYPPSSLGRAITDGQAGFGNDTLSKVPGISSIEQGMTGLKIGGDLTAAVTKTVGTALSSSGMTSIATNSVPPVSSAAPKPTSWAAIARKPAKPQPKLKPKGNVGIGGSAVPPPPIKHNMNIGTWDEKGSVVKAPPTQPVLPPQTIIQQPQPLIQPPPLVQSQLPQQQPQPPQPQQQQGPQPQAQPHQVQPQQQQLQNRWVAPRNRGAGFNQNNGAGSENFGLGVVPVSASPSSVEVHPVLEKLKAINNYNPKDFDWNLKNGRVFIIKSYSEDDIHRSIKYSIWCSTEHGNKRLDAAYRSLNGKGPLYLLFSVNGSGHFCGVAEMKSVVDYNAYAGVWSQDKWKGKFEVKWIFVKDVPNNQLRHIRLENNDNKPVTNSRDTQEVPLEKAKQVLKIIATFKHTTSIFDDFAHYEKRQEEEEAMRRERNRNKQ

SEQ ID NO: 7 - DNA, 40 bp, Homo sapiens
  misc_feature (1)..(40): RHOB-C1
cccctgccggcacggctccccctcctggaccagtcccccg

SEQ ID NO: 8 - DNA, 32 bp, Homo sapiens
  misc_feature (1)..(32): ASH1L
ccaggaactatcactacagtaggactgctaag

SEQ ID NO: 9 - DNA, 37 bp, Homo sapiens
  misc_feature (1)..(37): RHOB-C2
acgttttcctttaaagacattggcagagttgggctag

SEQ ID NO: 10 - DNA, 56 bp, Homo sapiens
  misc_feature (1)..(56): JUN
acttttcgttaacattgaccaagaactgcatggacctaacattcgatctcattcag

SEQ ID NO: 11 - DNA, 26 bp, Homo sapiens
  misc_feature (1)..(26): GPBP1-C2
tccaacattgactttcttcatcactg

SEQ ID NO: 12 - DNA, 28 bp, Homo sapiens
  misc_feature (1)..(28): TBP
ctcttccactcacagactctcacaactg

SEQ ID NO: 13 - DNA, 48 bp, Homo sapiens
  misc_feature (1)..(48): BTBD7
aaactgacttgacttttgggctgactcctaacagaccttcactttctg

SEQ ID NO: 14 - DNA, 39 bp, Homo sapiens
  misc_feature (1)..(39): GPBP1-C1
tccaacattgactttcttcatcactgcaacatttctctg

SEQ ID NO: 15 - DNA, 41 bp, Homo sapiens
  misc_feature (1)..(41): UBE2L3
aacaagacggacttcctctcccttcggactcacagcctttg

SEQ ID NO: 16 - RNA, 5 nt, Artificial Sequence
  Synthetic m6A Sequence 1
ggacu

SEQ ID NO: 17 - RNA, 5 nt, Artificial Sequence
  Synthetic m6A Sequence 2
agacu

SEQ ID NO: 18 - RNA, 5 nt, Artificial Sequence
  Synthetic m6A Sequence 3
ugacu

SEQ ID NO: 19 - RNA, 5 nt, Artificial Sequence
  Synthetic m6A Sequence 4
gaacu

SEQ ID NO: 20 - RNA, 5 nt, Artificial Sequence
  Synthetic m6A Sequence 5
aaacu

SEQ ID NO: 21 - RNA, 5 nt, Artificial Sequence
  Synthetic m6A Sequence 6
uaacu

SEQ ID NO: 22 - RNA, 5 nt, Artificial Sequence
  Synthetic m6A Sequence 7
ggacc

SEQ ID NO: 23 - RNA, 5 nt, Artificial Sequence
  Synthetic m6A Sequence 8
agacc

SEQ ID NO: 24 - RNA, 5 nt, Artificial Sequence
  Synthetic m6A Sequence 9
ugacc

SEQ ID NO: 25 - RNA, 5 nt, Artificial Sequence
  Synthetic m6A Sequence 10
gaacc

SEQ ID NO: 26 - RNA, 5 nt, Artificial Sequence
  Synthetic m6A Sequence 11
aaacc

SEQ ID NO: 27 - RNA, 5 nt, Artificial Sequence
  Synthetic m6A Sequence 12
uaacc

SEQ ID NO: 28 - RNA, 5 nt, Artificial Sequence
  Synthetic m6A Sequence 13
ggaca

SEQ ID NO: 29 - RNA, 5 nt, Artificial Sequence
  Synthetic m6A Sequence 14
agaca

SEQ ID NO: 30 - RNA, 5 nt, Artificial Sequence
  Synthetic m6A Sequence 15
ugaca

SEQ ID NO: 31 - RNA, 5 nt, Artificial Sequence
  Synthetic m6A Sequence 16
gaaca

SEQ ID NO: 32 - RNA, 5 nt, Artificial Sequence
  Synthetic m6A Sequence 17
aaaca

SEQ ID NO: 33 - RNA, 5 nt, Artificial Sequence
  Synthetic m6A Sequence 18
uaaca

SEQ ID NO: 34 - protein, 580 aa, Unknown
  Goat YTHDF2
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFASETLNKAPGMNTIDQGMAALKLGSTEVASNVPKVVGSAVGSGSITSNIVASNSLPPATIAPPKPASWADIASKPAKQQPKLKTKNGIAGSSLPPPPIKHNMDIGTWDNKGPVAKAPSQALVQNIGQQPTQGSPQPVGQQANNSPPVAQASVGQQTQPLPPPPPQPAQLSVQQQAAQPTRWVAPRNRGSGFGHNGVDGNGVGQTQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 35 - protein, 558 aa, Unknown
  Cat YTHDF2
MDLSLLNFNTVFEKNRLLLNLRNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFASETLNKAPGMNTIDQGMAALKLGSTEVASNVPKVVGSAVGSGSITGNIVASNSLPAATIAPPKPASWADIASKPAKQQPKLKTKNGIAGSSLPPPPIKHNMDIGTWDNKGPVAKAPSQALVQNLGQQPTQGSPQPVGQQANNSPPVAQASVGQQTQPLPPPPPQPSQLSVQQQATQPTRWVAPRNRGSGFGHNGVDGNGVGPSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 36 - protein, 580 aa, Unknown; identical to SEQ ID NO: 34
  Zebu YTHDF2

SEQ ID NO: 37 - protein, 579 aa, Unknown
  Gray mouse YTHDF2
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASNVPKVVGSAVGSGSITSNIVASSSLPPATIAPPKPASWADIASKPAKQQPKLKTKNGIAGSSLPPPPIKHNMDIGTWDNKGPVAKAPSQALVQNIGQPTQGSPQPVGQQANNSPPVAQASVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 38 - protein, 579 aa, Unknown
  Beaver YTHDF2
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASSVPKVVGSAVGSGSITSNIVASSSLPPATIAPPKPASWADIASKPAKQQPKLKTKNGIAGSSLPPPPIKHNMDIGTWDNKGPVAKAPSQALVQNIGQPAQGSPQPVGQQANNSPPVAQTSVGQQTQPLPPPPPQPAQLSVQQQAAQPTRWVAPRNRGSGFGHNGVDGNGVGQSQAGSGSAPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 39 - protein, 579 aa, Unknown
  Rat YTHDF2
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASSVPKVVGSAVGSGSITSNIVASSSLPPATIAPPKPASWADIASKPAKQQPKLKTKNGIAGSSLPPPPIKHNMDIGTWDNKGPVAKAPSQALVQNIGQPTQGSPQPVGQQANNSPPVTQTSVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 40 - protein, 580 aa, Unknown
  Consensus YTHDF2
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASNVPKVVGSAVGSGSITSNIVASNSLPPATIAPPKPASWADIASKPAKQQPKLKTKNGIAGSSLPPPPIKHNMDIGTWDNKGPVAKAPSQALVQNIGQQPTQGSPQPVGQQANNSPPVAQASVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK